(19) United States
(12) Patent Application Publication
(10) Pub. No.: US 2005/0143630 A1
(43) Pub. Date: Jun. 30, 2005

(54) System and method of testing cognitive function

(75) Inventors: David G. Darby, West Melbourne (AU); Ashley Bush, Somerville, MA (US); Paul Maruff, Ivanhoe (AU); Alex Collie, Kensington (AU)

Correspondence Address: PILLSBURY WINTHROP SHAW PITTMAN LLP, ATTENTION: DOCKETING DEPARTMENT, 11682 EL CAMINO REAL, SUITE 200, SAN DIEGO, CA, 92130, US

(73) Assignee: CogState, Ltd, Carlton (AU)

(21) Appl. No.: 11/064,539

(22) Filed: Feb. 23, 2005

Related U.S. Application Data
(63) Continuation of application No. 10/144,437, filed on May 10, 2002, now abandoned.
(60) Provisional application No. 60/317,639, filed on Sep. 6, 2001.
(60) Provisional application No. 60/317,571, filed on Sep. 6, 2001.

(30) Foreign Application Priority Data
May 11, 2001 (AU) PR 4924
Aug. 10, 2001 (AU) PR 6948
Sep. 5, 2001 (AU) PR 7516

(51) Int. Cl. A61B 5/00

(52) U.S. Cl. 600/300

(57) ABSTRACT

A system and method of diagnosing the onset and monitoring the progression of cognitive impairment may incorporate administering one or more psychological tests and instructing a subject regarding rules for responding to the one or more tests without providing cultural cues such as may be introduced in language-based instruction techniques. Proper test responses may be simulated during an instruction phase preceding the testing phase. An apparatus, system, and method of testing cognitive function may be implemented in a computerized system.

### CROSS-REFERENCE TO RELATED APPLICATIONS

The present application claims the benefit of provisional application Ser. No. 60/317,639, filed Sep. 6, 2001, entitled “DIAGNOSIS AND MONITORING OF MINIMAL PROGRESSIVE COGNITIVE IMPAIRMENT,” and of provisional application Ser. No. 60/317,571, filed Sep. 6, 2001, entitled “TREATMENT OF MINIMAL PROGRESSIVE COGNITIVE IMPAIRMENT.”

### BACKGROUND

1. Field of the Invention

Aspects of the present invention relate generally to testing cognitive function, and more particularly to a system and method of diagnosing the onset and monitoring the progression of cognitive impairment and analyzing the efficacy of treatments therefore.

2. Description of the Related Art

Serious cognitive impairments and dementias represent an increasing percentage of disability cases diagnosed and treated worldwide; the number of dementia patients may be expected to increase, particularly as global life expectancies increase and the population ages. Alzheimer's Disease (AD), which is estimated to affect 10% of the population over the age of 65 and 50% of the population over the age of 80, is typically considered the most common of the myriad possible causes of dementia. Other forms of dementia include vascular dementia, dementia with Lewy body formation, fronto-temporal dementia, post-traumatic dementia, human immuno-deficiency virus (HIV) associated dementia, atypical dementias, Parkinsonism, Huntington's disease, and toxicity resulting from substance abuse or adverse drug effects.

Currently, AD and other dementias are usually not diagnosed until one or more warning symptoms have already appeared. At their earliest manifestation, these symptoms may constitute a syndrome known as Mild Cognitive Impairment (MCI), which was recently defined by the American Academy of Neurology; MCI refers to the clinical state of individuals who have memory impairment when compared with age appropriate normative data, but who are otherwise functioning well, and do not meet clinical criteria for dementia (see, e.g. Petersen, R. C., Stevenas, J. C., Ganguli, M., Tangalos, E. G., Cummings, J. L., & DeKosky, S. T., Practice parameter: Early Detection of Dementia: Mild Cognitive Impairment. Neurology 56 1133-1142 (2001)).

It is generally accepted that MCI is a precursor of AD in about 50% of documented cases. Additionally or alternatively, MCI may also be a precursor of dementias resulting from other pathological causes. Such alternative causes of MCI can be difficult to differentiate clinically from AD when the MCI itself is first diagnosed.

MCI may be detected using conventional cognitive screening tests such as the Mini Mental Status Exam, the Memory Impairment Screen, and various other neuropsychological screening batteries; if performance results fall outside the range of accepted normative data, MCI may be diagnosed. These diagnostic methods are inadequate as set forth below.

Until relatively recently, treatment for conditions involving cognitive deficits was generally not available; once a diagnosis of such a condition was made, deterioration towards dementia was typically considered an inevitable consequence. Only supportive care was possible. A variety of cognitive enhancers have recently become available. While these enhancers generally do not address the underlying pathology causing AD and other cognitive dysfunction, they appear to be fairly effective in slowing cognitive deterioration.

Moreover, intensive research into the causes of AD has led to the development of a number of putative therapeutic agents, for example: monoclonal antibody directed against amyloid protein; clioquinol or other metal chelators; protease inhibitors; anti-oxidants; adduct breaking agents; growth factors; anti-inflammatory agents; oestrogens; or statins. The current availability of several therapeutic methods suggests-that early diagnosis of conditions leading to dementia is of great importance; treatment should begin before the damage caused by the condition is so great that it causes actual disability.

Conventional diagnostic methodologies for degenerative cognitive conditions employ tests which are designed or optimized to be administered only once; if administered more than once, traditional tests may show large practice effects based upon changes in the strategies employed by the tested subjects. That is, the tested subjects may develop strategies to improve performance with respect to typical testing methods.

As noted above, in accordance with existing systems and methods of cognition evaluation, deterioration of cognitive function has already begun by the time any symptomatic deficiencies may be detected.

### SUMMARY

In accordance with one aspect of the present invention, for example, a method of evaluating cognitive function comprises administering a test operative to diagnose cognitive impairment; and instructing a subject regarding rules for the test without providing cultural cues such as language-based instructions. Testing may be selectively repeated.

Similarly, a method of administering a sequence of tests generally comprises selecting test comprising a plurality of test trials and operative to diagnose a condition of cognitive impairment; instructing a subject regarding rules for responding to the plurality of test trials without providing cultural cues; administering the test; recording responses to a plurality of test trials displayed during test administration; and selectively repeating the foregoing operations for an additional test.

A finding that a particular test or test sequence result indicates measurable degradation in cognitive function relative to reference result or previously recorded response data may be indicative of pre-symptomatic cognitive impairment.

It will be appreciated that the foregoing methods may be suitable for monitoring the efficacy of a therapeutic agent or other treatment regimen. In some embodiments, the methods may further include treating the subject's pre-symptomatic cognitive impairment condition prior to obtaining a further test result and determining whether the test result has changed.

Accordingly, a method of evaluating the efficacy of a treatment regimen for treating cognitive impairment generally comprises: selecting a test operative to evaluate cognitive function; instructing a subject regarding rules for the test without providing cultural cues; administering the test; recording responses to a plurality of test trials displayed during test administration; measuring a condition of cognitive impairment; treating the subject in accordance with a treatment regimen; selectively repeating the test; and evaluating the treatment regimen using a comparison of results obtained during successive iterations of the test.

Treatment may involve treating the subject with a cognitive enhancer such as a cholinesterase inhibitor, for example: Aricept; Exelon; Reminyl; and Cognex. Such enhancers are currently available for symptomatic treatment of conditions such as AD, and several other enhancers are in pre-clinical or clinical trial. Additionally or alternatively, treatment may involve treating the subject with an agent directed at correcting a causative mechanism of AD, such as monoclonal antibody directed against amyloid protein, clioquinol or other metal chelators, protease inhibitors, growth factors, anti-oxidants, adduct breaking agents, anti-inflammatory agents, oestrogens, or statins.

Cognitive functions tested may include memory, speed at memory tasks, decision-making, concentration, attention, and problem-solving; cognitive function scores may be based on speed and accuracy measurements.

In some embodiments, a method of testing cognitive function may preclude a subject from enhancing performance, speed, or accuracy through practice or repetition; accordingly, a subject cannot learn to ‘beat’ the test through strategy or otherwise.

A tested subject may produce a reference result by performing a test multiple times. The test may be performed over a wide range of time intervals, depending upon the purpose; for example, in order to differentiate between an impaired and a non-impaired group of subjects, the test may be administered three or four times on the same day or in rapid succession. To monitor progression of cognitive impairment or to evaluate the efficacy of treatment, the same or similar test may be administered at intervals of three to six months, for example.

A test of cognitive function may evaluate the memory of the subject in order to produce a measure of the subject's memory function related to the subject's accuracy at performing memory tasks. The measure of the subject's memory function may also relate to the subject's speed in performing memory tasks.

A test of cognitive function may generally comprise a plurality or battery of discrete tests for evaluating or quantifying memory aspects of cognitive impairment. The battery of tests may be presented in a standard format, allowing indices which bridge a number of tests to be extracted.

A test of cognitive function may also evaluate the decision-making, concentration, attentional, and problem solving functions of the subject. Diagnosis may involve comparing test response data to a reference test data set; the comparison result may be used to determine any deterioration of the foregoing or other cognitive functions.

Pre-symptomatic cognitive impairment may represent a marker of a condition which is a precursor of progressive cognitive decline such as caused by AD, vascular dementia, dementia with Lewy body formation, fronto-temporal dementia, post-traumatic dementia, HIV-associated dementia, atypical dementia, Parkinsonism, Huntington's disease, or toxicity resulting from substance abuse or adverse drug effects. Additionally or alternatively the pre-symptomatic cognitive impairment, per se, may be such a condition.

In some instances, pre-symptomatic cognitive impairment may be characterized as “minimal” progressive cognitive impairment (MPCI).

In some embodiments, some or all of the foregoing methods may be used in conjunction with other methods of diagnosing or monitoring cognitive impairment. For example, it is has been reported that impairment of the sense of smell is a characteristic symptom of the very early stages of AD; a non-invasive diagnostic test of olfactory function is currently available. Other tests for early symptoms are also available, for example, based upon detection of neural thread protein.

Various embodiments of the present invention present a significant advantage in detecting pre-symptomatic cognitive impairment. Specifically, a system and method of testing cognitive impairment allow pre-symptomatic cognitive impairment and MPCI to be detected more reliably and more certainly; additionally, such conditions may be diagnosed more rapidly, in terms of serial study, than has hitherto been possible.

In accordance with other aspects of the invention, for example, systems, apparatus, and computer readable media are employed to execute or to implement the described methods. An apparatus or system operative to evaluate cognitive impairment generally comprises a testing module operative to administer a test and an instruction module operative to instruct a subject regarding rules for the test without providing cultural cues. Such an apparatus or system may include a data structure operative to store responses and data related thereto; additionally or alternatively, a data transmission interface may enable or allow communication with a remote device via a network. In some embodiments, the foregoing operation may be controlled or supervised by a test coordinator module.

### BRIEF DESCRIPTION OF THE DRAWINGS

The foregoing and other aspects of various embodiments of the present invention will be apparent through examination of the following detailed description thereof in conjunction with the accompanying drawings.

FIG. 1A is a simplified diagram illustrating a data communication network environment in which one embodiment of a psychological testing system may be employed.

FIG. 1B is a simplified diagram illustrating components of the embodiment depicted in FIG. 1A.

FIG. 2 is a simplified block diagram illustrating one embodiment of a psychological testing apparatus.

FIG. 3 is a simplified block diagram illustrating components of one embodiment of a psychological testing apparatus.

FIG. 4A is a simplified flow diagram illustrating the general operation of one embodiment of a psychological testing method.

FIG. 4B is a simplified flow diagram illustrating the general operation of one embodiment of a psychological testing method facilitating administration of a test sequence.

FIG. 5 is a simplified flow diagram illustrating the general operation of one embodiment of a method of instructing a test subject.

FIG. 6 is a simplified flow diagram illustrating the general operation of one embodiment of a method of performing a test.

FIG. 7 is a simplified diagram illustrating one embodiment of a trial time line.

FIG. 8 is a simplified flow diagram illustrating the general operation of one embodiment of a psychological diagnostic method.

FIG. 9 is a simplified flow diagram illustrating the general operation of one embodiment of a method of ascertaining the efficacy of a treatment regime.

FIG. 10 is a simplified diagram illustrating one embodiment of a graphical user interface for a system and method of testing cognitive function.

FIG. 11 is a simplified diagram illustrating a start configuration displayed by a system and method of testing cognitive function.

### DETAILED DESCRIPTION

Embodiments of the present invention overcome the foregoing and various other shortcomings of conventional technology, providing a system and method of testing cognitive function and identifying the onset and progression of cognitive impairment.

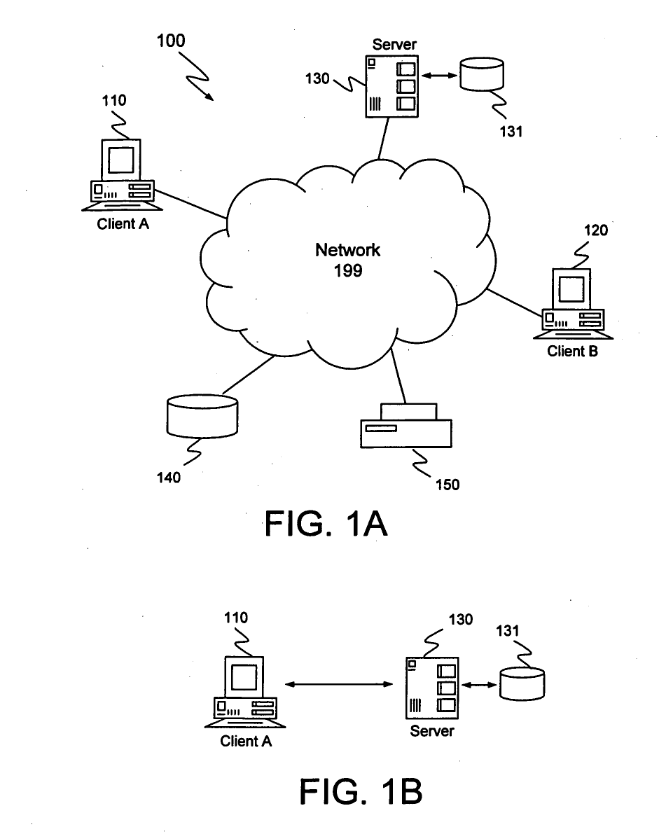

Turning now to the drawings, FIG. 1A is a simplified diagram illustrating a data communication network environment in which one embodiment of a psychological testing system may be employed. In the exemplary FIG. 1A embodiment, system 100 generally comprises one or more remote computers or terminals, such as network clients 110 and 120, coupled to one or more servers, such as server 130, via a communications network 199. System 100 may also comprise data storage media and peripheral equipment, represented by reference numerals 140 and 150, respectively.

For clarity, only one server 130 and two clients 110, 120 have been depicted in FIG. 1A. Those of skill in the art will appreciate that the arrangement illustrated in FIG. 1A is presented for illustrative purposes only, and that system 100 may be implemented with any number of additional servers, clients, or other components; the number and variety of each device coupled to network 199 may vary in accordance with system requirements. In some embodiments, the functionality of one device, such as peripheral device 150, for example, may reside on or be enabled by another device, such as server 130.

In operation, clients 110, 120 may be capable of two-way data communication via communications network 199. In that regard, client 110 may communicate with client 120, server 130, peripheral device 150, and data storage medium 140 via network 199 or via one or more additional networks (not shown) which may be coupled to network 199. It will be appreciated by those of skill in the art that clients 110, 120, server 130, and other components depicted in FIG. 1A may be coupled via any number of additional networks without inventive faculty.

In some embodiments, clients 110, 120 may be personal computers or workstations, personal digital assistants (PDAs), wireless telephones, or other network-enabled computing devices, electronic apparatus, or computerized systems. In operation, clients 110, 120 may execute software or other programming instructions encoded on a computer-readable storage medium, and additionally may communicate with server 130, data storage medium 140, and peripheral device 150 for monitor and control applications. For example, client 110 may interrogate server 130 and request transmission of data maintained at data storage medium 131 coupled to, or accessible by, server 130. Additionally or alternatively, client 110 may transmit control signals or requests which may cause device 150 to take some action or to execute a specified function or program routine.

It is well understood in the art that any number or variety of peripheral equipment, such as device 150, may additionally be coupled to network 199 without departing from the essence of the present disclosure. Examples of such peripheral devices include, but are not limited to: servers; computers; workstations; terminals; input/output devices; laboratory equipment; printers; plotters; routers; bridges; cameras or video monitors; sensors; actuators; or any other network-enabled device known in the art. Peripheral device 150 may be coupled to network 199 directly, as illustrated in FIG. 1A, or indirectly, for example, through server 130, such that the functionality or operation of device 150 may be influenced or controlled by hardware or software resident on server 130.

As is generally known in the art, server 130 may be embodied or implemented in a single physical machine, for example, or in a plurality of distributed but cooperating physical machines. In operation, server 130 may incorporate all of the functionality of a file server or application server, and may additionally be coupled to data storage medium 131. Accordingly, information and data records maintained at data storage medium 131 may be accessible to clients 110, 120 through bidirectional data communication with server 130 via network 199.

Network 199 may be any communications network known in the art including, for example: the internet; a local area network (LAN); a wide area network (WAN); a Virtual Private Network (VPN); or any system providing data communication capability between clients 110, 120, server 130, storage medium 140, and peripheral device 150. In addition, network 199 may be configured in accordance with any topology known in the art, including star, ring, bus, or any combination thereof.

By way of example, the data connection between components in FIG. 1 may be implemented as a serial or parallel link. Alternatively, the data connection may be any type generally known in the art for communicating or transmitting data across a computer network; examples of such networking connections and protocols include, but are not limited to: Transmission Control Protocol/Internet Protocol (TCP/IP); Ethernet; Fiber Distributed Data Interface (FDDI); ARCNET; token bus or token ring networks; Universal Serial Bus (USB) connections; and Institute of Electrical and Electronics Engineers (IEEE) Standard 1394 (typically referred to as “FireWire”) connections.

Other types of data network interfaces and protocols are within the scope and contemplation of the present disclosure. In particular, clients 110, 120 may be configured to transmit data to, and receive data from, other networked components using wireless data communication techniques, such as infrared (IR) or radio frequency (RF) signals, for example, or other forms of wireless communication. Accordingly, those of skill in the art will appreciate that network 199 may be implemented as an RF Personal Area Network (PAN).

Storage media 131 and 140 may be conventional read/write memory such as magnetic disk drives, magneto-optical drives, optical disk drives, floppy disk drives, compact-disk read only memory (CD-ROM) drives, digital versatile disk read only memory (DVD-ROM) drives, digital versatile disk random access memory (DVD-RAM) drives, transistor-based memory, or other computer-readable memory devices for storing and retrieving data.

FIG. 1B is a simplified diagram illustrating components of the embodiment depicted in FIG. 1A. The components in the FIG. 1B arrangement may generally incorporate all of the respective functionality set forth above. Responsive to requests or instructions from client 110 as set forth below, for example, server 130 may be operative to retrieve data or information from storage medium 131. Storage medium 131 may comprise a database, for instance, or other data structure and may be configured to maintain software code, files, data, and the like required for conducting cognition analysis in whole or in part.

Accordingly, methods of diagnosing the onset and monitoring the progression of cognitive impairment, as well as methods of analyzing the efficacy of treatments for cognitive deficiencies, may be performed by computer executable instructions or other program code resident at client 110, server 130 and storage medium 131, or a combination thereof.

In some embodiments, for example, software code resident at client 110 may be configured to perform a battery of interactive tests designed to diagnose cognitive impairment or to measure the progression of cognitive dysfunction; diagnostic or prognostic data, or information representative of that data, may be transmitted to server 130 via a data communication network is indicated in FIG. 1A. Additionally or alternatively, some or all of the test functionality may be incorporated in software code resident at server 130; in such an embodiment, for example, test data or results may be transmitted in whole or in part to client 110 via a network.

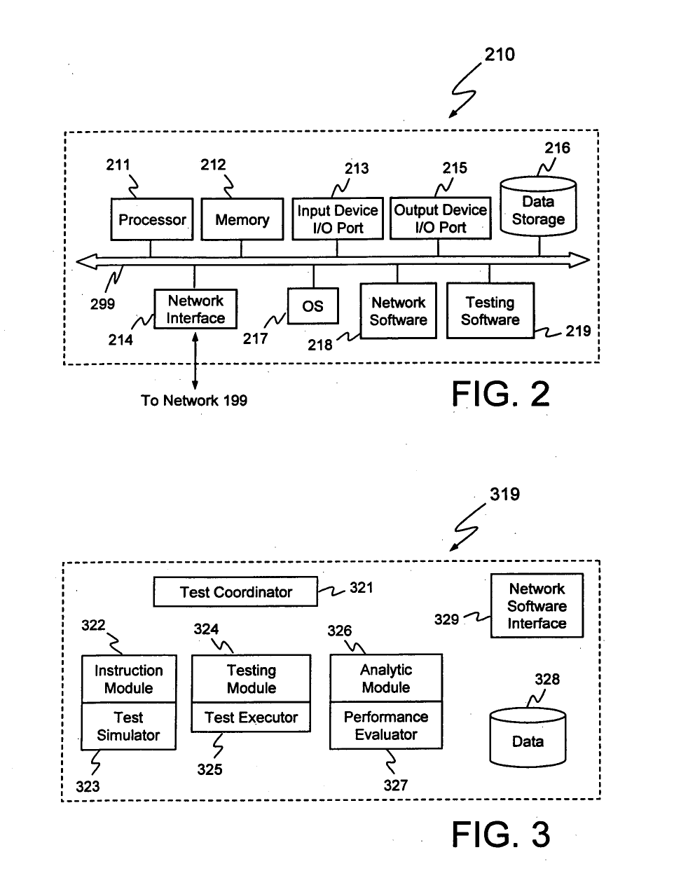

FIG. 2 is a simplified block diagram illustrating one embodiment of a psychological testing apparatus. The simplified testing apparatus 210 depicted in FIG. 2 may generally correspond to network client 110 illustrated and described above with reference to FIGS. 1A and 1B. In that regard, apparatus 210 may be embodied in the various types of devices noted above and incorporate all of the functionality and operational characteristics set forth in detail above. It will be appreciated that apparatus 210 may also be implemented as an isolated system, i.e. not coupled to a network. Accordingly, apparatus 210 may be embodied in a computer workstation or desktop computer, for example, and may be configured to run a multi-tasking operating system (OS) 217 as is generally known in the art.

As indicated, the FIG. 2 embodiment may generally comprise a processor 211, memory 212, and data storage medium 216 coupled to a system bus 299. As is generally known in the art of computerized systems, operation of the foregoing and other elements of apparatus 210 may be influenced or controlled by OS 217. Input device port 213 and output device port 215 generally enable bi-directional data communication between apparatus 210 and various peripheral devices known in the art.

Processor 211 may be any microprocessor or microcontroller known in the art. Software code or programming instructions for controlling the functionality of processor 211 may be encoded in memory 212 or stored in storage medium 216. Memory 212 and storage medium 216 may be any computer-readable memory known in the art. Additionally or alternatively, some software or instruction code related to operation of processor 211 may reside at a remote server 130 or storage medium 131 accessible through network 199, as described above with reference to FIGS. 1A and 1B. A network interface 214 may enable the foregoing network communication, and may be any interface known in the art, or developed and operative in accordance with known principles, for communicating or transferring files across a computer network.

Processor 211 may communicate via bus 299 with a plurality of peripheral equipment, including network interface 214, for example, enabling two-way network data communications as described above. In that regard, network software 218 may provide appropriate networking protocols and data formats as described above to enable network data transfer in accordance with system requirements.

Peripheral devices configured and operative to communicate with computerized systems are well known in the art; such equipment may include a display or a speaker (not shown) coupled to output device port 215, a manual input device or a microphone (not shown) coupled to input device port 213, and the like. In some embodiments, apparatus 210 may be coupled to a visual display such as a cathode ray tube (CRT) monitor, a liquid crystal display (LCD) screen, a touch-sensitive screen, or other monitor device known in the art for displaying images and text. Similarly, apparatus 210 may be coupled to a manual input device such as a conventional keyboard, keypad, mouse, trackball, or other input device. It will be appreciated that apparatus 210, some or all of the foregoing devices, or a combination thereof, may include digital-to-analog and analog-to-digital conversion circuitry, as appropriate.

In operation, apparatus 210 may execute program instructions or software code, represented by testing software 219, configured and operative to evaluate cognitive abilities or degradation thereof. Testing software 219 may operate in conjunction with data records, profile data, and the like maintained at data storage medium 216 to provide diagnostic or prognostic results of cognitive function. In some embodiments set forth in more detail below, cognitive function may be measured or evaluated through interactive testing procedures during which input is received via input device port 213; the input may generally be responsive to output such as visual stimuli, for example, displayed or otherwise presented via output device port 215.

As noted briefly above, testing software 219 or various components thereof may be resident on more than a single physical machine. While the FIG. 2 embodiment illustrates testing software 219 resident at apparatus 210, the present disclosure is not intended to be limited in any way by the FIG. 2 illustration. It will be appreciated by those of skill in the art that the networked configuration of apparatus 210 enables some or most of the functionality of testing software 219 to reside elsewhere, such as at server 130 as described above, for example. The extent to which the functionality of testing software 219 may be implemented at a network client such as apparatus 210 may be a function of, among other things, the current processing load and overall capabilities of processor 211 and memory 212, the clock speed of bus 299, the bandwidth of network 199 to which network interface 214 is coupled, and so forth. Distributed load processing and application functionality is known in the art.

FIG. 3 is a simplified block diagram illustrating components of one embodiment of a psychological testing apparatus. The testing software 319 depicted in FIG. 3 generally corresponds to testing software 219 illustrated and described above with reference to FIG. 2. Testing software 319 generally comprises an instruction module 322, a testing module 324, and an analytic module 326, the operation of which may be managed or coordinated by a test coordinator module 321. A network software interface 329 may facilitate communication between testing software 319 and network software to enable data communication with a remote server or other device as described above with reference to FIG. 2.

Additionally, testing software 319 may incorporate or have access to a data storage medium 328, which may be embodied in a database, library file, or other suitable data structure; data maintained at data medium 328 may be directly or indirectly related to cognitive testing methods, results, analysis, and the like. For example, normative data related to average test results for a particular population or test control group may be stored in data medium 328 to facilitate comparisons with received test responses. Historic and current test response data and information derived therefrom may also be stored in data medium 328 either permanently, for future analysis or comparisons, or temporarily, pending transmission to a remote device for review and analysis.

Test coordinator module 321 may organize and manage all testing operations. In that regard, modules 322, 324, and 326 may be configured to transmit interim results or ongoing progress to test coordinator 321, which may monitor and evaluate progression through an individual test or a particular sequence of test procedures comprising a battery of tests. Test coordinator 321 may additionally communicate network communications requirements to network software interface 329, facilitating distributed testing. Accordingly, test coordinator 321 may control test sequences occurring either locally or on a global scale.

Instruction module 322 may be configured to provide useful instructions regarding test procedures and the manner in which a test subject is expected to respond to test conditions. In the FIG. 3 embodiment, instruction module 322 comprises a test simulator 323 operative to provide a simulation of the current test and to illustrate correct responses to various test stimuli or test trials. In operation, test simulator 323 may provide visual cues indicative of test procedures and proper methods of response to a plurality of test trials. In that regard, test simulator 323 may provide instruction by example, and therefore may omit written or other language-based instruction paradigms.

In embodiments employing instruction module 322, control of testing sequences may pass to testing module 324 upon completion of appropriate instruction procedures. In some embodiments, for example, test coordinator 321 may be apprised (by instruction module 322) of completion of one or more test simulations, and may then initiate software code or other executable instructions or routines at testing module 324 which enable test executor 325 to commence a particular testing operation. Test executor 325 may present test trials and record response data (in data medium 328, for example) in accordance with predetermined test protocols.

Analytic module 326 may be responsive to instructions or control signals from test coordinator 321, and may be operative to initialize and perform analytic operations involving test responses and other data received from testing module 324, for example. Additionally or alternatively, some functionality of analytic, module 326 may be incorporated into testing module 324, and may facilitate performance of analysis tasks in parallel with test operations, i.e. test responses and related data may be analyzed as they are received during testing procedures executed by test executor 325.

In any event, a performance evaluator 327 associated with analytic module 326 may interpret test response data and information derived therefrom. In some embodiments, normative, characteristic, or historic data records maintained at data medium 328 may be compared with current test responses and data acquired by test executor 325. Depending upon overall system configuration and requirements, test response data and other information may be fully analyzed by performance evaluator 327 or transmitted to a remote device for additional analysis; such transmission may be facilitated by network software interface 329 as set forth above.

As noted above, testing software 319 may generally be distributed across one or more physical machines, depending, for example, upon system requirements, processing capabilities, and local or system-wide load characteristics. In some embodiments, for example, test coordinator 321 may reside on a network client, while most or all of the other components illustrated in FIG. 3 may reside on a remote server. Those of skill in the art will appreciate that the FIG. 3 embodiment is provided by way of example only, and that various system software configurations are possible.

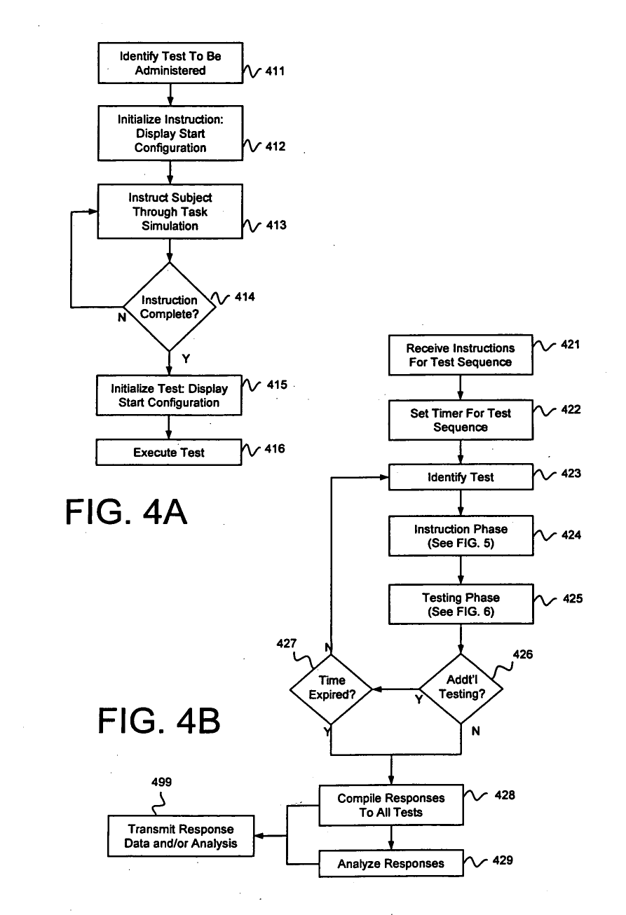

FIG. 4A is a simplified flow diagram illustrating the general operation of one embodiment of a psychological testing method. As indicated in FIG. 4A, a particular test to be administered may be identified as represented at block 411. In some embodiments, for example, particularly in cases where a plurality of discrete tests are administered in series, this identification may be performed by a test coordinator such as illustrated and described above with reference to FIG. 3; additionally or alternatively, a specific test may be identified and selected through interaction with an icon, a menu, a file list, or other selectable element typically presented on a computer display as part of a graphical user interface (GUI).

An instruction phase of the test may be initialized as indicated at block 412; the instruction phase may generally be controlled by an instruction module as set forth above with reference to FIG. 3. A ‘start,’ or initial, configuration may be displayed on a computer display, for example. Such a start configuration may represent the general layout or organization of the items or stimuli which will embody individual test trials during testing operations. By way of example, a deck of playing cards or a number of ordinary dominoes may be displayed in a particular arrangement relative to each other. The number, type, and orientation of the items displayed in the start configuration, as well as their relative locations and the overall arrangement of graphical elements represented, may generally be a function of the particular test to be administered.

A test subject may be instructed by example or through task simulations as represented at block 413. In particular, an instruction module may include a test simulator as set forth in detail above with reference to FIG. 3. In the FIG. 4A embodiment, such a test simulator may illustrate a plurality of test trials and simulate correct responses thereto; in that regard, the test simulator may additionally provide visual or other cues operative to instruct a test subject with respect to appropriate interaction with one or more input devices. Accordingly, an instruction module and test simulator may provide instructions regarding the test which follows without resort to written or other language-based feedback.

By way of example, a test may be designed to evaluate a subject's responses to the random or pseudo-random display of ordinary playing cards; an exemplary test may require a particular reaction responsive to display of cards from the red suits (diamonds and hearts) and a different reaction responsive to display of black cards (spades and clubs). In this example, a start configuration may comprise a view of a deck of cards depicted face-down and a stylized image of a computer keyboard, mouse, or other device required to input responses. Simulation of a test trial may comprise a graphical representation of a card, selected from the top of the face-down deck, being turned over to reveal its value and suit. An indication of the proper reaction or response may be displayed in conjunction with the particular simulated test trial.

In the example above, for instance, a proper response to a red card may be selection of a particular key on a computer keyboard (e.g. the ‘R’ key), while a proper response to a black card may be selection of a different key (e.g. the ‘B’ key). During instruction through simulation as represented by block 413, each simulated test trial may be accompanied by an indication of the proper response thereto. If a red card is selected from the top of the simulated deck, the ‘R’ key may be highlighted or otherwise emphasized in the displayed image of a keyboard; similarly, the ‘B’ key may be highlighted responsive to a black card being selected from the top of the deck. Employing ‘R’ to represent red and ‘B’ to represent black may introduce a language-based bias during instruction; accordingly, it may be desirable to require a response to a particular card color using a key (e.g. ‘K’ for red and ‘D’ for black) which has no intrinsic language associations. As noted above, an appropriate response key may be highlighted or emphasized when a card is displayed during the instruction through simulation.

It will be appreciated that instruction through task simulation depicted at block 413 may take various forms, depending upon the complexity of the administered test and the proper responses required for various test trial events; different types of responses and illustrations thereof are contemplated. For example, a proper response to a test trial may include selection of a certain mouse button; simulated test trials requiring this response may be accompanied by an illustration of a mouse having the appropriate button highlighted, for example, or a graphical representation of a finger depressing the proper mouse button. Various methods of illustrating or highlighting elements of input devices are known in the art.

As noted above, an administered test may include various trials, each of which may require a particular response. As represented by decision block 414, an instruction phase may comprise an iterative loop, repeating test simulation until all of the possible test trials and their respective appropriate responses for a given test are simulated and illustrated. In the foregoing example, for instance, at least two iterations may be necessary to simulate the two possible test trial events, i.e. a red card or a black card. In some embodiments, instruction through task simulation as represented by block 413 and decision block 414 may continue until each type of test trial event is simulated a predetermined number of times.

At block 415, instruction has been completed (as determined at decision block 414) and the test to be administered is initialized. A start configuration, representative of the beginning of the test, may be displayed; as set forth above, a start configuration may represent the general layout or organization of the items or stimuli which will embody individual test trials. Additionally, an indication of the proper response to test trials may be included in the start configuration display. In some embodiments described in more detail below, a representation or indication of the proper response for test trials may be provided during the test phase until a predetermined number of correct responses is achieved.

As indicated at block 416, following test initialization and display of the start configuration, the selected test may be executed. Initialization and execution of the test (blocks 415 and 416, respectively) may be performed by a testing module including a test executor as illustrated and described in detail above with reference to FIG. 3. Referring to both FIGS. 3 and 4A, for example, test coordinator 321 may pass control of the testing sequence from instruction module 322 to testing module 324 responsive to a signal indicating that instruction has been completed (block 414); such a signal from instruction module 322 may initiate program code at test coordinator 321, which in turn, may initialize the test (block 415) and invoke test executor 325 to administer the test (block 416) in accordance with predetermined testing objectives and protocols.

In accordance with the FIG. 4A embodiment, instruction initialization (block 412) and execution (blocks 413 and 414), as well as test initialization (block 415) and execution (block 416), are generally performed with respect to a particular test which may be identified or selected (block. 411) independently of any testing sequence or battery of multiple tests. In some instances, however, it may be desirable to administer a plurality of tests in sequence.

FIG. 4B is a simplified flow diagram illustrating the general operation of one embodiment of a psychological testing method facilitating administration of a test sequence. As indicated at block 421, a test apparatus or system may receive instructions identifying a plurality or battery of discrete tests to be administered in sequence. By way of example, a test coordinator 321 may receive instructions from a local processor, for example, or from a remote server or client, requesting or instructing that a particular test sequence be administered. The received instructions may comprise testing protocols or directions; additionally or alternatively, the received instructions may simply direct test coordinator 321 to retrieve necessary testing information and protocol data from a specified data source or address, for example, such as data medium 328 illustrated in FIG. 3.

In some embodiments, a testing sequence may comprise a time limit for administrative, logistical, or other reasons. In such instances, it may be appropriate to set a clock or timer mechanism as indicated at block 422; it will be appreciated that the timer set in the FIG. 4B embodiment may represent a global timing device for the entire test sequence. Additionally or alternatively, each discrete test which is a component of the test battery or sequence may include one or more time limits. For example, a response for each test trial may be limited to a predetermined time frame, while the total time allotted for completion of a specified number of trials in a single test may similarly be limited. As indicated in FIG. 4B, the total time allotted for completion of the plurality of tests in a given testing sequence may likewise be selectively limited as desired.

While time for the test sequence has not expired (as measured at decision block 427) and every test in the test sequence has not been completed (as determined at decision block 426), blocks 423 through 427 illustrate an iterative approach to completing the sequence of tests in the selected test battery. A test to be completed (the first or subsequent test in the sequence) may be identified at block 423, which may generally correspond to the operation at block 411 described above.

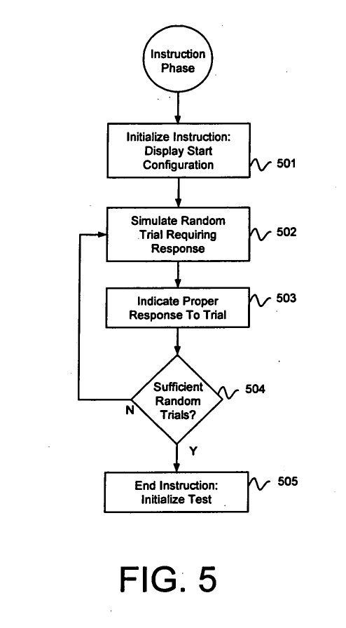

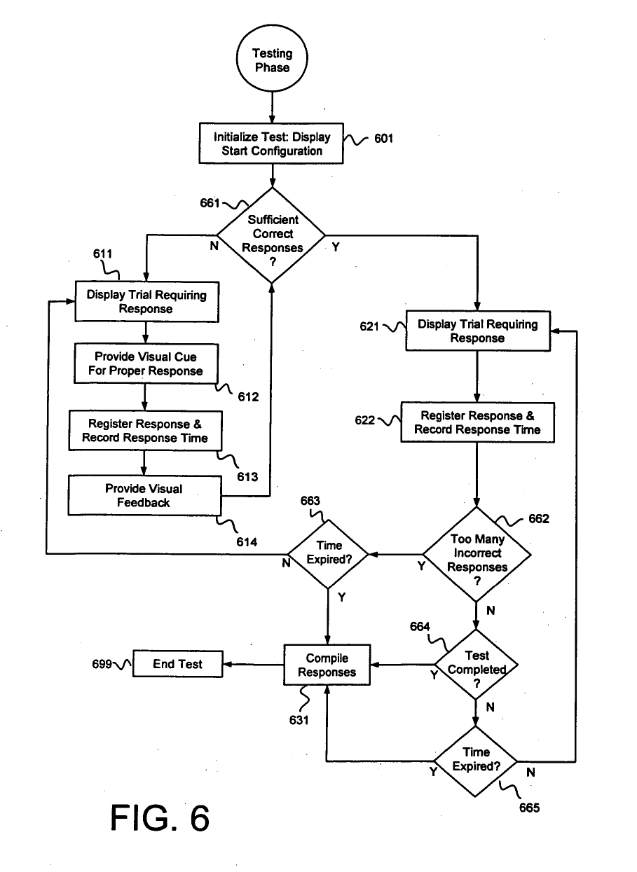

The instruction phase (block 424) for each individual test in the sequence generally corresponds to blocks 412-414, and is described in more detail below with reference to FIG. 5. Similarly, the testing phase (block 425) for each test in the sequence generally corresponds to blocks 415 and 416, and is described in more detail below with reference to FIG. 6.

Upon completion of all the tests in the sequence as determined at decision block 426, or upon expiration of the timer as measured at decision block 427, responses to all the test trials may be compiled at block 428. Trial responses, data related to aspects of the responses, and information derived from both may be analyzed as indicated at block 429; additionally or alternatively, response data and information derived therefrom may be transmitted to a remote device (as indicated at block 499) for initial or additional analysis. The extent to which trial responses and data representative of the responses are analyzed prior to transmission may depend upon processor capabilities at the local machine, data transmission bandwidth, security or privacy concerns, and the like.

FIG. 5 is a simplified flow diagram illustrating the general operation of one embodiment of a method of instructing a test subject. Aspects of the FIG. 5 embodiment were described above with reference to FIG. 4A. It will be appreciated that a method such as illustrated in FIG. 5 may be incorporated into the instruction phase (block 424) in FIG. 4B. As indicated at block 501, instruction may be initialized and a start configuration may be displayed as set forth above.

The start configuration generally represents the organization of visual stimuli embodying individual test trials; such visual stimuli may include ordinary or stylized playing cards, dominoes, or other visual representations of identifiable objects. As noted above, the various stimuli may be displayed in a unique arrangement dependent upon the selected test protocol.

At block 502, a test subject may be instructed by example through random task simulations. In particular, a test simulator may illustrate a particular test trial (block 502) and simulate a correct response thereto as represented at block 503; the test simulator may provide visual or other cues indicative of proper interaction with one or more input devices required to input the correct response.

Returning to the red and black playing card example described above with reference to FIG. 4A, display of a particular card (the ace of spades, for instance) represents a simulated random test trial event at block 502. In this example, highlighting or otherwise emphasizing the correct keyboard key or mouse button, for example, provides an indication of the proper response input for the test trial event (block 503); in this example, the ‘H’ key (for example) may be highlighted in the image of a keyboard, indicating that depressing the ‘H’ key is an appropriate response when a black card is displayed.

As represented by decision block 504, an instruction phase may comprise an iterative loop, repeating test simulation (blocks 502 and 503) until all of the possible test trial events and their respective appropriate responses are simulated and illustrated. Returning to the example above, a second iteration may display a red card (the queen of hearts, for example). In conjunction with display of such a red card, a test simulator may highlight the ‘A’ key (for example) in the image of a keyboard, illustrating the proper response when a red card is displayed.

As noted above, reinforcement of instruction through task simulation may continue until each type of test trial event is simulated a predetermined number of times. In the red and black card test, for example, instruction may not be clear with only one iteration for a black card and one iteration for a red card. Reinforcement through sufficient iterations may solidify the test rules, and facilitate understanding of protocols for the selected test.

At block 505, instruction is complete and the test to be administered may be initialized. Control of a single test operation (FIG. 4A) may proceed to block 415, for example, while control of a multiple test sequence (FIG. 4B) may proceed to block 425. In some embodiments described in more detail below, a representation or indication of the proper response for test trial events may be provided during at least a portion of the test phase, e.g. until a predetermined number of correct responses is recorded.

FIG. 6 is a simplified flow diagram illustrating the general operation of one embodiment of a method of performing a test. It will be appreciated that a method such as illustrated in FIG. 6 may be incorporated into the testing phase (block 425) in FIG. 4B. Following test initialization and display of the start configuration at block 601, the selected test may be executed. As set forth in detail above with reference to FIG. 3, initialization and execution of the test may be performed by a testing module including a test executor, both of which may comprise software code or other computer-executable instructions.

The start configuration displayed at block 601 may illustrate the organization of the items or stimuli (such as playing cards or dominoes, for example) which will embody individual test trials. Additionally, an indication of the proper response to test trials may be included in the start configuration display. As noted above, a representation or indication of the proper response for test trials may be provided during at least a portion of the test phase; in some embodiments, such prompting or indication of proper responses may continue until a predetermined number of correct responses has been input.

Accordingly, as indicated at decision block 661, a method of performing a test may monitor the number of correct responses and compare that number with a predetermined threshold as defined by protocols for the particular test being administered. The threshold number of correct responses may be based upon consecutive correct responses, for example, or simply a total number of correct responses, irrespective of any intervening incorrect responses.

In an alternative embodiment, the evaluation of correct responses at decision block 661 may be replaced by a timer for example, such that illustration or simulation of correct responses ceases after a predetermined or random period of time.

In the FIG. 6 embodiment, a determination that a threshold number of correct responses has not been reached may be interpreted as an indication that additional instruction is necessary; accordingly, the testing procedure may pass to block 611. A test trial requiring a response may be displayed at block 611, along with a graphical or other representation of the input device required for response. At block 612, a visual cue indicating the correct response to the test trial event may also be provided; the visual cue may be similar to that provided during the instruction phase. As noted above, visual instruction cues for the test trial event displayed at block 611 may include highlighting or otherwise identifying the proper input mechanism (such as a keyboard key or mouse button, for example) on the representation of the input device.

A response to the test trial event may be recorded along with associated information at block 613. As indicated in the FIG. 6 embodiment, response time may be recorded in conjunction with the response; correct and incorrect responses, as well as associated response times, may be compiled and analyzed together or separately as appropriate.

A visual indication of the appropriateness of the recorded response may be provided in the form of feedback, as indicated at block 614. In some embodiments, visual feedback may be accompanied or replaced by aural or other perceptible cues. In this portion of the testing protocol, one goal is to establish that the test subject understands the rules and procedures of the test; accordingly, the operation at block 614 may provide sufficient feedback to reinforce accurate responses and to discourage incorrect input.

A determination that a threshold number of correct responses has been reached or surpassed may be interpreted as an indication that additional instruction is unnecessary; accordingly, the testing procedure may pass to block 621. As indicated in the flow diagram, a test trial requiring a response may be displayed at block 621; during this portion of the test, however, visual cues or instructions representing a correct input response are omitted.

At block 622, a response to the test trial event may be recorded along with associated information such as response time, for example.

At decision block 662, a determination that the test subject has input a sufficient number of incorrect responses may be interpreted as an indication that additional instruction is required. Accordingly, too many incorrect responses may result in the test procedure returning to block 611 if the time allotted or allowed for the test has not expired (as determined at decision block 663). The evaluation at decision block 662 may be based upon consecutive incorrect responses, for example, or simply a total number of incorrect responses, irrespective of any intervening correct responses; as with the determination at decision block 661, a threshold number of incorrect responses as measured at block 662 may be a function of testing protocols.

If a threshold number of incorrect responses has not be reached, a method of performing a test in accordance with the FIG. 6 embodiment may determine if the test is complete at decision block 664. Depending upon testing protocols, completion of a particular test may require recordation of a threshold number or percentage of correct responses, for example, or require a predetermined number of test trials. If the test is not complete, the test procedure may return to display the next test trial at block 621 if the time allotted for the test has not expired (as determined at decision block 665).

Upon completion of the test or expiration of the timer, for example, response data and associated information may be compiled as indicated at block 631 and the test may end (block 699). During administration of a multiple test sequence or battery such as in the embodiment illustrated in FIG. 4B, for example, the test procedure may proceed to decision block 426.

It will be appreciated that in a single test embodiment of the method illustrated in FIG. 6, various alternatives and modifications are within the scope and contemplation of the present disclosure. For example, compilation of results at block 631 may be a continuous process, for instance, and may occur during recordation of response data and other information at blocks 613 and 622. Additionally, analysis and transmission of response data and associated information may occur prior to or subsequent to the end of the test at block 699.

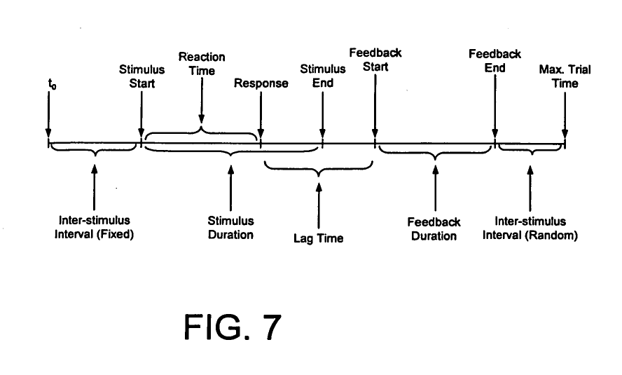

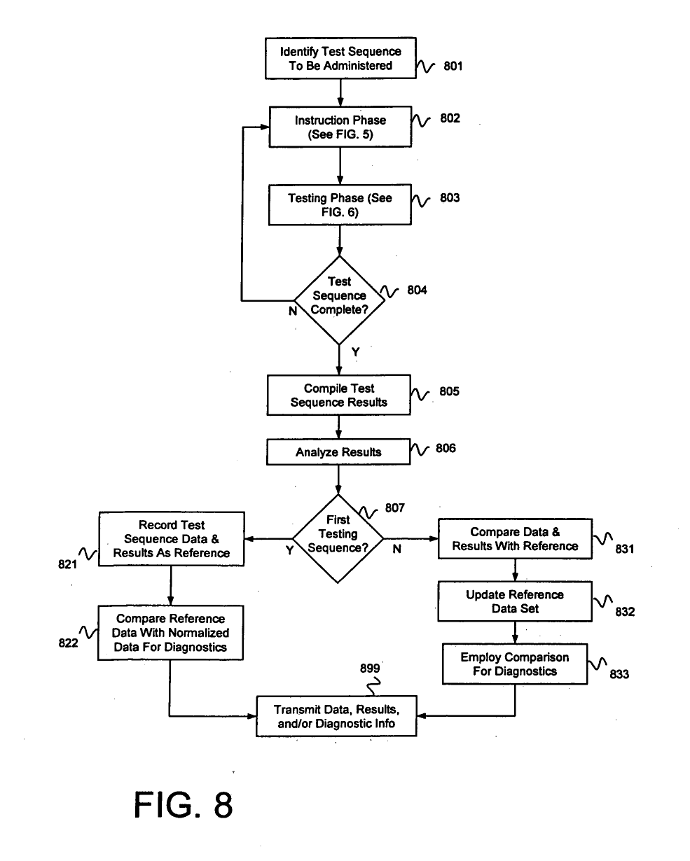

FIG. 7 is a simplified diagram illustrating one embodiment of a trial time line. The various events depicted in the exemplary trial time line of FIG. 7 may be associated with the respective operations illustrated in blocks 611-614 or blocks 621 and 622 in FIG. 6, for example.

In that regard, a test executor such as described above may employ a test trial algorithm for structuring a test trial as indicated in FIG. 7. The algorithm underlying the test trials may be sufficiently flexible to accommodate different testing paradigms and protocols; accordingly, the FIG. 7 embodiment may establish test trials which satisfy widely varying stimulus presentation and test requirements.

As described above with reference to FIGS. 4-6, a test may generally comprise multiple trials, each of which may include component parts; different component parts may be active at different stages in the trial time line. A trial settings or profile data record may store information related to one or more trial time line criteria; such settings or profile data may be maintained at a data storage medium 328 such as illustrated and described above with reference to FIG. 3. This data may be accessible to the test executor or trial structure algorithm mentioned above.

By way of example, a trial engine or trial structure algorithm implemented at the test executor may monitor boolean flags to determine which part of the time line has been reached. When a timed interval elapses or a specific time horizon is reached, a corresponding function or software procedure may be initiated, enabling the test executor to determine the implementation details which are appropriate and consistent with the testing protocol.

Specific intervals measured from initiation of a trial (designated to in FIG. 7) may be common among all trials, though the duration of each such interval may vary in accordance with test protocols and other factors.

A fixed inter-stimulus interval (ISI) may represent a fixed period of time between to and the beginning of the trial stimulus (stimulus start); this fixed ISI may be constant (i.e. “fixed”) across all trials during a particular administration of a particular test. It will be appreciated that the value or duration of the fixed ISI may vary between tests or between different administrations of the same test.

A minimum reaction time (RT) filter may be implemented, generally restricting the earliest time for which a valid response may be recorded, i.e. any response input detected prior to this minimum RT may be designated an “anticipatory response” and may be ignored. For example, an input received prior to the stimulus start may be anticipatory, since any such input is clearly not responsive to the test trial event. As an alternative to ignoring or omitting such responses from test results or data analysis, anticipatory responses may be considered as potentially indicative of disease or other cognitive impairment; in that regard, every key depression or missed trial event may be recorded for subsequent analysis.

A reaction time may be measured from the stimulus start to detection of a valid response; as noted above, reaction time responsive to test trials may provide important data related to cognitive function. In the FIG. 7 embodiment, a response time may be measured provided that the maximum time duration for the test trial has not expired.

Stimulus duration may be measured from the stimulus start to the stimulus end. In some embodiments, such as illustrated in FIG. 7, for example, stimulus duration may be a fixed or predetermined time period; accordingly, stimulus duration may be constant across all trials during a particular administration of a particular test, irrespective of the reaction time for a given test trial. Alternatively, stimulus duration may be modified depending upon test design and protocol, and may generally vary in accordance with reaction time.

Specifically, the stimulus end for a particular trial may coincide with or immediately follow the time of the response; accordingly, the stimulus duration illustrated in FIG. 7 may be substantially equal to the reaction time. Additionally, in accordance with some test protocols, for example, feedback may begin immediately upon termination of the trial stimulus, i.e. the feedback start may coincide with or immediately follow the stimulus end, which in turn may coincide with the response input. Those of skill in the art will appreciate that such a test trial time line structure may increase the number of trials which are possible in a given period of time by compressing the lag period between recordation of a response and commencement of feedback; additionally or alternatively, compressing the lag time illustrated in FIG. 7 may shorten the total duration of a given test.

Further, while a feedback duration is indicated in FIG. 7 and discussed above, it will be appreciated that some test procedures may not require a feedback portion of the trial time line. For example, the testing operations illustrated and described above with reference to blocks 621 and 622 of FIG. 6 may omit feedback by design; accordingly, the feedback duration for test trials under such circumstances may be set to zero.

A random inter-stimulus interval (ISI) may be measured from the end of the feedback (if any) to the end of the trial; the random nature of the random ISI may be test dependent or trial dependent. In some instances, for example, the random ISI for every trial in a given test may be identical, though the value may be randomly selected at the beginning of the test; alternatively, the random ISI may be selected or determined at random for each individual trial during a given test. The duration of the random ISI may be determined at run time, and may vary from zero time to a given maximum duration (e.g. 1000 ms).

Each trial may be limited to a maximum trial duration, which is defined as the maximum allowable elapsed time from t₀ to the end of the trial.

In some embodiments of a trial time line, response input may be received at any time during the trial. As noted above, input prior to the minimum RT may result in the response being designated anticipatory. For input occurring subsequent to the stimulus start but prior to maximum trial time, a valid reaction time may be recorded; if such input occurs subsequent to the feedback start, any response may be designated “post-feedback,” which may affect interpretation when test results are compiled and analyzed. In some embodiments operative in accordance with the principles illustrated in FIG. 7, multiple anticipatory and post-feedback responses may be measured, but only one reaction time may be recorded.

As noted above, the time elapsed between stimulus start and feedback start may not be fixed, since this duration may be dependent upon when and whether a response is input. The total duration of the FIG. 7 trial is guaranteed to be no greater than the maximum trial time; it will be appreciated that the trial may be as short as the sum of the reaction time, any feedback duration (if not zero), and the fixed and random ISIs. While any or all the intervals may be set zero by the test executor or test trial algorithm, if all are zero or set below minimal human perception thresholds, the trial may occur so quickly as to be ineffective. Various goals and test objectives may dictate appropriate intervals for the FIG. 7 embodiment.

FIG. 8 is a simplified flow diagram illustrating the general operation of one embodiment of a psychological diagnostic method. It will be appreciated that the operations of identifying a test sequence (block 801), instructing and testing (blocks 802 and 803, respectively) with respect to discrete tests in the test sequence, compiling test results (block 805), and analyzing test data and associated information (block 806) may generally correspond to the testing embodiment illustrated and described in detail above with reference to FIG. 4B. Decision block 804 and the loop back to block 802 represent the iterative nature of a test battery or sequence comprising multiple discrete testing procedures.

At decision block 807, the method of FIG. 8 may determine if prior test results have been obtained for a particular test subject. If the current test sequence is the first such battery of tests completed by the test subject, the diagnostic procedure may proceed to block 821, where response data, results, and associated information may be recorded. Such data may be stored as one or more data records in a database maintained, for example, at medium 328 or another accessible data storage medium. Recordation of reference data from a first testing sequence completed by a particular test subject may facilitate subsequent comparisons with additional test results obtained during successive test sequences completed by the same individual. Additionally, some or all of the data and information collected during a first test sequence may be employed in creating or augmenting normalized population data sets.

As indicated at block 822, reference data obtained through a first test sequence may be compared with normalized or characteristic data; the comparison may be used for diagnostic inferences. It will be appreciated that normalized or characteristic data sets may represent average, expected, ‘normal,’ or mean testing results for test subjects falling into certain categories or satisfying specified profile criteria Among other factors, age, gender, education level, documented head injuries, habitual use of prescribed or recreational drugs, various personality traits, and the like may all influence construction and application of such normalized data sets as contemplated in the FIG. 8 embodiment. As noted above, a reference data set for a particular test subject may be compared with recorded normalized data for diagnostics and general evaluation.

It will be appreciated that the operations illustrated at blocks 821 and 822 may occur substantially simultaneously; for example, a comparison with corresponding records from the normalized data set may be made as each response datum is written to memory. Alternatively, comparison at block 822 may occur prior to recordation at block 821.

If previous test sequence results are stored as reference data for a particular test subject, for example, the diagnostic procedure may continue as indicated at block 831. In this instance, response data, results, and other information from the current test sequence (blocks 801-806, for example), may be compared with reference data recorded at an earlier time or date. In some embodiments, a reference data set to be used in subsequent comparisons may be updated (as indicated at block 832) with response data and results obtained during the most recent test sequence. As indicated at block 833, the foregoing comparison may facilitate diagnosis of cognitive impairment or disorder in accordance with test protocols and diagnostic paradigms.

In addition to, or as an alternative to, the comparison operations at blocks 822 and 831, response data and test sequence results may be transmitted to a remote device as indicated at block 899 for initial or further analysis. Where diagnostic procedures and data manipulation are conducted as represented at blocks 822, 831, and 833, any resulting data or other comparison information related to the diagnosis may also be transmitted to a remote device at block 899. The relative emphases on local data processing and data transmission for distributed processing may depend in large part upon system hardware configuration, network bandwidth, and other factors as set forth in more detail above.

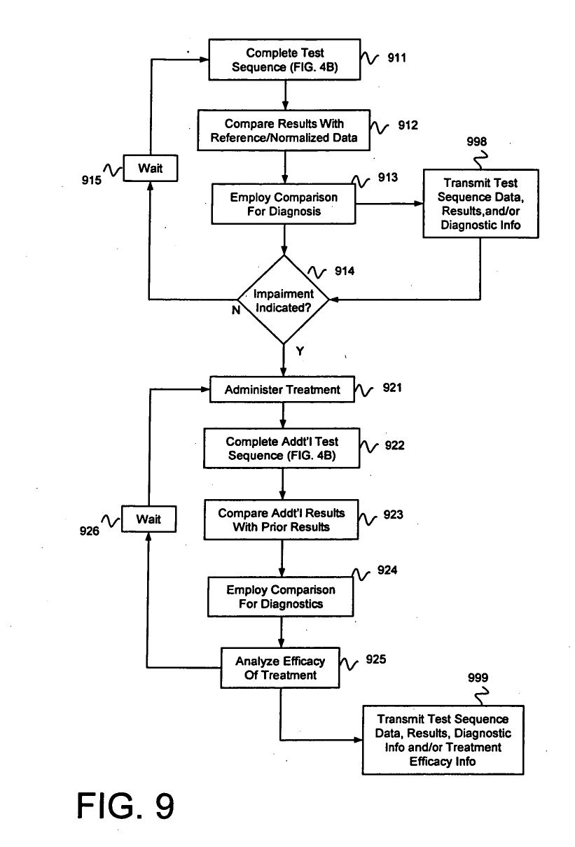

FIG. 9 is a simplified flow diagram illustrating the general operation of one embodiment of a method of ascertaining the efficacy of a treatment regime. The operations illustrated at blocks 911-913 generally correspond to descriptions set forth above. In the FIG. 9 embodiment, one or more test sequence results may determine whether cognitive impairment is indicated (block 914) based upon a comparison of test sequence data with either a normative test data set or a previously obtained reference data set; in some embodiments, the comparison at block 913 may be similar to the comparisons described above with reference to FIG. 8, for example. Response data, test results, and intermediate diagnostic information may be transmitted (as indicated at 998) to a remote device such as a dedicated computer server or work station, for instance, for initial or further analysis facilitating the determination at decision block 914.

Where cognitive impairment is not indicated, the FIG. 9 embodiment may return to block 911 after an appropriate interval (block 915). For example, a period of 6-18 months may elapse between administration of test sequences as illustrated at the top of FIG. 9; the wait period at block 915 may be customized to match the needs of a particular test subject, and may be a function of specific risk factors for cognitive impairment, current state of cognitive functionality, family medical history, and the like. In some embodiments, for example, test sequences may be administered more frequently than the 6-18 month interval noted above; it may be beneficial or desirable to administer one or more test sequences monthly, weekly, or daily under some circumstances, depending upon, inter alia, some or all of the foregoing factors.

Where cognitive impairment is indicated at decision block 914, a method of ascertaining the efficacy of treatment may include treating the test subject; accordingly, treatment may be administered as indicated at block 921. Various types and methods of treatment for numerous types of cognitive impairment or dysfunction are set forth above. Following completion or progression through at least a part of a treatment regimen, an additional test sequence may be completed as indicated at block 922.

Additional testing (block 922), comparison of test results with normative or reference data sets (block 923), and diagnostic analysis of comparison results (block 924) may generally correspond with the operations described above in detail with reference to FIGS. 4B and 8. The comparison of the most recent test results with normative or reference data sets may provide an indication of the efficacy of the treatment as illustrated at block 925.

It is generally accepted in the art that cognitive impairment is expected to progress in such a manner as to be increasingly debilitating. The analysis at block 925 may seek to determine a rate of degradation for cognitive functionality relative to control group data or other standardized references. For example, slower than expected cognitive decline during or following treatment at block 921 may be related to successful or efficacious treatment regimens, while normal or increased rate of decline for cognitive functions may be indicative of less effective treatments. Those of skill in the art will appreciate that myriad factors may influence the determination at block 925, as well as the initial diagnosis of cognitive impairment at block 914. The present disclosure is not intended to be limited by any empirical, experimental, clinical, or other diagnostic methods represented at decision block 914 or block 925, nor are the treatments which may be administered at block 921 intended to be interpreted in any limiting sense.

An exemplary method of evaluating the efficacy of cognitive treatment as illustrated in FIG. 9 may return to block 921 after an appropriate interval (block 926). For example, a period of 6-18 months may elapse between administration of test sequences. Alternatively, a test sequence may be administered every twenty minutes over the course of several hours or an entire day, for example. A wait period at block 926 may be customized to the needs of a particular test subject, and may be a function of any or all of the following factors: family medical history; specific risk factors for cognitive impairment; current state of cognitive impairment; rate of diagnosed cognitive decline; typical duration of the treatment regime and any rehabilitation time; and the like. The foregoing list is representative of some factors which may influence the time period indicated at block 926; the list is not intended to be exhaustive.

It will be appreciated that various alternatives or modifications may be implemented with respect to the method embodiments, and that the presented order of the individual blocks is not intended to imply a specific sequence of operations to the exclusion of other possibilities. The particular application and overall system requirements may dictate the most efficient or desirable sequence of the operations set forth in FIGS. 4-6, 8, and 9.

Those of skill in the art will appreciate that the foregoing embodiments facilitate initial diagnosis and monitoring of treatment for very early cognitive deterioration of the kind which may be expected in the prodromal phases of AD. In the following description, this early state of cognitive decline or pre-symptomatic condition is referred to as minimal progressive cognitive impairment (MPCI). The system and method of testing cognitive impairment described herein provide a computerized cognitive screening apparatus and methodologies adapted to detect and to monitor the progression of MPCI.

It will be clearly understood that the following description is not intended to be limited to AD and its congeners; the principles of early detection apply equally to all conditions associated with progressive cognitive impairment, including but not limited to fronto-temporal, atypical or HIV dementia, Parkinsonism, Huntington's disease, toxicity resulting from substance abuse, and adverse drug effects. Furthermore, where abnormalities are detected using the system, apparatus, and methods illustrated and described above with reference to FIGS. 1-9, further investigation may be required in order to determine one or more specific causes of the abnormal test results.

In some embodiments, the tests illustrated in FIG. 4-9 may be designed so as to be repeatable using equivalent alternative forms. Accordingly, the tests may facilitate maximum performance based exclusively upon the relative ability of the test subject. Any improvement in performance reflects only the subject's capability with respect to performing the test, since improvement beyond physiological limits of speed and response accuracy is not possible. That is, the exemplary tests described below may be designed such that a particular test subject cannot develop strategies to improve performance based upon familiarity with the mechanics of responding to test trial events.

As noted above, decision-making, concentration, and problem-solving skills, as well as any noticeable deterioration thereof, may provide further indication of a cognitive impairment. Additionally, a battery of tests having a standard or common format (such as playing cards, for example) may be designed to evaluate different aspects of cognitive function while eliminating or reducing any potential bias due to test format differences; conversely, if tests employing different formats are used within a given test sequence, bias or anomalies in the results may be caused by differences in the formats of the individual tests in the battery.

Furthermore, the system and method of testing cognitive impairment described herein facilitate tests or test batteries in which cultural influences, such as language skills, for example, may be eliminated from the test results or reduced significantly. As set forth in detail above, instruction or direction with respect to performing a test may be provided by example or test simulation, without the need for language-based instructions. Alternatively, language-based instructions may optionally be provided, at least during an instruction phase such as illustrated in FIG. 5; although such verbal or written instructions may initially influence test performance and results, allowing the subject to re-test until optimization (i.e. the subject is fully familiar with the rules, protocols, and mechanics of the test) may remove or substantially reduce any residual negative effects due to language barriers, miscommunication, or misunderstanding.

The various methods set forth above may facilitate monitoring the status of subjects with recognized or diagnosed cognitive impairments; importantly, re-testing may enable an accurate measure of the rate of deterioration or improvement of the subject's cognitive function. In that regard, the efficacy of treatment regimens for cognitive impairment may be evaluated and monitored as described above with reference to FIG. 9. Methods developed in accordance with the exemplary embodiment may be employed to screen putative drugs for the treatment or prevention of pre-symptomatic cognitive impairment. A subject diagnosed with cognitive impairment may be administered a putative drug using an appropriate treatment regimen, and then re-tested; as set forth above, a system and method of testing cognitive impairment may ascertain whether the rate of decline has decreased, halted, or reversed.

Characterization of MPCI

As noted briefly above, MPCI may be characterized as a prodromal or pre-clinical syndrome; as used herein, MPCI is generally characterized by the following clinical criteria:
the patient or test subject exhibits no significant cognitive symptoms;
the patient or test subject is functionally independent in activities of daily living;
informants familiar with the patient or test subject do not report apparent cognitive difficulties; in that regard, an informant may or may not be aware of the test subject's present impairment or risk of future decline;
the test subject's performance on objective cognitive tests falls generally within the normal range, based on any single test administration (i.e. performance cannot be differentiated from normal subjects using cross-sectional evaluation); and
the test subject shows progressive deterioration on serial testing with the system and methods described herein.

As noted above, a test battery may be provided with multiple equivalent alternate forms. Informative psychometric tests which are components of the present system and method may include at least the following relevant properties:
objective performance-based measures of speed and accuracy;
equivalence of stimuli throughout the test, e.g. exemplars drawn from finite sets of familiar stimuli such as game indicia (playing cards, dominoes, dice, chess pieces, and the like);
random stimulus selection from within the set of available stimuli;
multiple administrations of stimuli within each task, increasing statistical power;
minimal or no strategy-dependent practice effects to facilitate response optimization; and
broad-based sampling of cognitive domains including, for example, simple and complex attention, memory, and adaptive problem solving.

The foregoing testing properties may facilitate reliable serial assessment of cognitive performance and may minimize result errors or anomalies due to individual or test-related factors. Reliable detection of change may be influenced by optimal individual performance, limited only by neurophysiological ability. Accordingly, the exemplary tests described below may be designed to provide sufficient practice opportunities at each review session such that the subject may optimize performance at each test session.

In conventional neuropsychological tests, significant pre-assessment practice or performance optimization may invalidate inferences about the normality of performance based on comparisons with normative data ranges; such conventional neuropsychological tests are designed to be administered only once.

One of the characteristics of MPCI mentioned above is that the condition may be identifiable only by serial testing showing progressive decline in one or more cognitive functions. In addition, subtle changes may only be discernible after multiple observations, which may be made on the same day, for instance, or weeks or months apart. In individuals without any cognitive impairment, serial performance measures regress toward the mean of a distribution of normal scores. Thus for any individual, repeated normal performances markedly decrease the probability that the individual may be incorrectly classified as impaired (i.e. false positive diagnosis or Type I error). Tests comprising the foregoing properties even allow repeated testing on the same day to differentiate normal from abnormal individuals. Serial assessment also allows for the reliable calculation of MPCI within individual variability.

Detection of MPCI

The following discussion illustrates an example of the practical use of the present system and method in the detection of very mild decline in cognitive function. As is generally known in the art, a current conceptualization recognizes mild cognitive impairment (MCI) as a progressive decline in cognitive function, while age-associated memory impairment (AAMI) is generally recognized as an abnormal, but much less rapidly declining or even static, cognitive impairment. Those of skill in the art will appreciate that AAMI may not actually exist as a distinct cognitive impairment; in particular, the generally recognized characteristics of AAMI are based upon older neuropsychological methods which do not take into account their own limitations for serial assessment as outlined above. Accordingly, it is possible that AAMI really represents a mixture of normal subjects suffering no cognitive decline at all and some MPCI subjects who are actually declining.

An assessment of cognitive function may occur when some impairment in cognition is already suspected. As noted above, MPCI is defined as pre-symptomatic decline detectable only by serial testing.

At assessment, it is likely that an individual with MCI or with AAMI will show some performance in the borderline abnormal range. On the basis of this single assessment, however, it may be impossible to determine whether abnormal performance reflects MCI or AAMI. Furthermore, given the low reliability of most neuropsychological test instruments and techniques, any borderline abnormal performance might also be attributable to error. Accordingly, on the basis of such a single assessment, a clinician generally advises that performance is equivocal, and that reassessment should take place at a future date, for example, in six or twelve months. If the individual actually has some cognitive impairment, whether this is declining or static, the interim between testing represents time wasted in terms of planning patient care or implementing pharmacotherapies aimed at preventing disease progression.

In accordance with the foregoing embodiments, the characteristically poor ability of traditional neuropsychological tests to guide decisions regarding degradation of cognitive function may be rectified by:

(1) Increasing the Reliability of the Tests Used to Assess Cognitive Performance.

This may both minimize false positive classification when an individual is healthy and minimize false negative classification when the individual actually suffers from abnormal cognitive performance. Increases in reliability may be achieved by repeated testing, provided that practice effects on test performance may be eliminated or substantially reduced by the test methodology and protocols. By itself, a strategy of repeated test administrations may not differentiate whether MPCI is due to MCI or AAMI (if AAMI exists).

(2) Regular Prospective Assessment of Cognitive Function.

This may allow accurate determinations of whether cognitive function is really declining, irrespective of the initial level or the relative stability of any impairment in cognitive function. Objective evidence of significant cognitive decline may suggest that MPCI will lead to MCI, while very mildly declining or static but impaired performance may suggest, or possibly disprove, AAMI.

(3) The Availability of Objective Data from Before Memory Impairments were Suspected.

This may allow statistical comparisons of current and previous assessments or test data and results; such statistical data and comparison results may facilitate determinations regarding abnormality or rate of deterioration.

Importantly, MPCI or MCI may be identified even if an individual's performance on a given test within a test sequence or battery during assessment is within the normal range. Abnormality may be inferred from a significant reliable decline in cognitive performance over time. Provided that the cognitive test allows multiple or repeated testing sessions, detection of cognitive decline may occur even before an individual meets any clinical criteria for MCI; this is the MPCI syndrome.

Testing Protocols

As noted in detail above with reference to FIGS. 1-3, a testing apparatus may generally comprise computer hardware and program software or other computer executable instructions, and may be embodied in a computer workstation, a personal, laptop, or portable computer, and the like; the apparatus further may include or be coupled to a monitor or display and one or more input devices such as a keyboard and a mouse. It will be appreciated that in some embodiments, input and output functionality may be integrated into a single device such as a touch-sensitive screen, for example. The computer executable instructions may be preinstalled on the apparatus or downloaded from a network such as the internet, for example, in the form of a Java™ applet.

As set forth in detail above with reference to FIGS. 4 and 5, a method of testing a subject may provide instructions, displaying a simulation of various test trial events on the monitor or display and additionally displaying an indication of an appropriate response to each simulated test trial event; the subject may learn how to perform the test by observing the simulation.

In the illustrated embodiments, an apparatus such as depicted in FIG. 3 may only test one subject at a time. A given test may be designed and implemented to last about fifteen to twenty minutes. A test or test sequence may be initiated, for example, by selecting an associated application icon on the display.

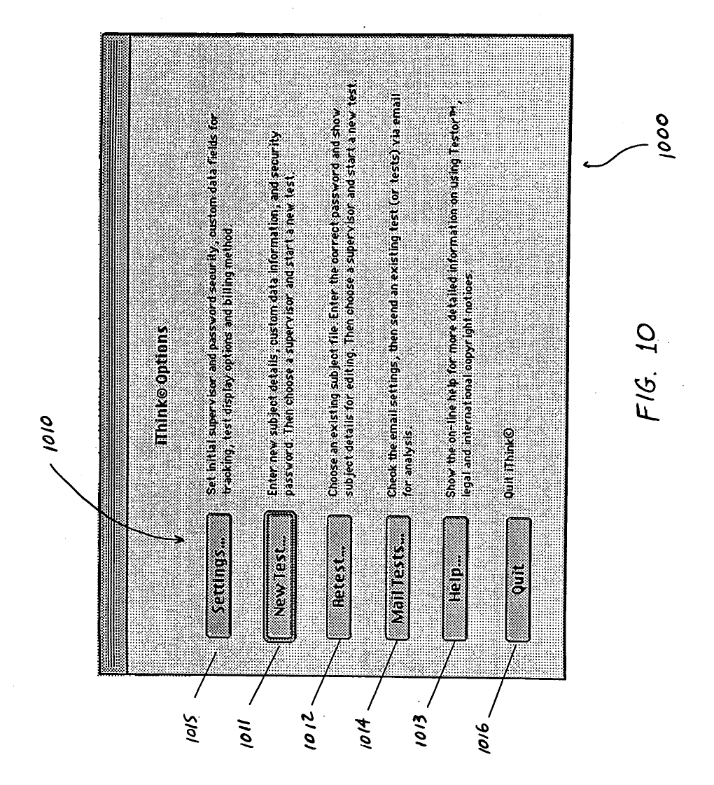

FIG. 10 is a simplified diagram illustrating one embodiment of a graphical user interface (GUI) which may be employed in conjunction with a system and method of testing cognitive function. A dialog box 1000 may include active icons or buttons 1010 representing available options when the program application is initialized. It will be appreciated that dialog box 1000 is provided by way of example only; variations and alterations may be made to the presentation of available options, as well as to the options themselves, depending upon system requirements and desired functional characteristics.

In the exemplary FIG. 10 embodiment, options may include starting a new test (“New Test . . . ” 1011), re-testing a subject who has already been tested at least once before (“Retest . . . ” 1012), viewing help information (“Help . . . ” 1013), transmitting completed test results for analysis (“Mail Tests . . . ” 1014), adjusting program settings or parameters (“Settings . . . ” 1015), or exiting the program immediately (“Quit” 1016). As is generally known in the art of computer interfaces, items represented in dialog box 1000 may have menu equivalents which may be selected to perform similar or equivalent actions without the need for interaction with dialog box 1000.

Each user or test subject may be provided with one or more data records (stored, for example, at data medium 328 in FIG. 3) related to personal or characteristic profile information. A standard “save file” dialog box as is generally known in the art may prompt entry of relevant, requested, or required profile data. Information which may be stored in a profile data record may include, but not be limited to, some or all of the following: name or some other identifier; title; company; home or business address; telephone numbers, electronic mail addresses, or other contact information; birth year; and the like. Additionally, characteristic information provided by a subject and stored as profile data may affect the testing methods, the analysis of test data and results, or both. Such information may include some or all of the following: gender; handedness; education level; and the like. Depending upon overall system requirements or institutional rules imposed by the company or firm administering the test, certain fields may be mandatory.

In some embodiments, it may be appropriate to provide password protection, encryption technology, or other measures to ensure confidentiality of private information. Test response data and associated information (such as response times, accuracy trends as measured with respect to time, and so forth) may be recorded in a log or other block of memory which is not accessible by the subject. Appropriate test data recording techniques will be apparent to those skilled in the art and further details are not provided herein.

An introductory screen of instructions, including a plurality of general or global (i.e. not test-specific) instructions, may provide a brief overview of the test method and an indication of appropriate responses which may be expected during the test administration; such an introductory screen may not explain how individual tests should be performed, since such test-specific explanations may be obtained during simulation of each particular test.

As set forth in detail above, each test in a test sequence or battery may generally comprise two distinct phases: a simulation or instruction phase, during which the test is illustrated to the subject; and a testing phase, during which the subject performs the test in accordance with the rules provided during the simulation. In the following examples, each test involves display of virtual playing cards as visual stimuli, though, as noted above, the present disclosure is not intended to be so limited.

Playing cards may be particularly appropriate stimuli for use in conjunction with a system and method of testing cognitive function, since playing cards are generally a cultural and also contain a number of levels of information. The exemplary tests may involve different presentations or orientations of the playing cards on the display, depending in part upon the different aspects of cognition to be evaluated.

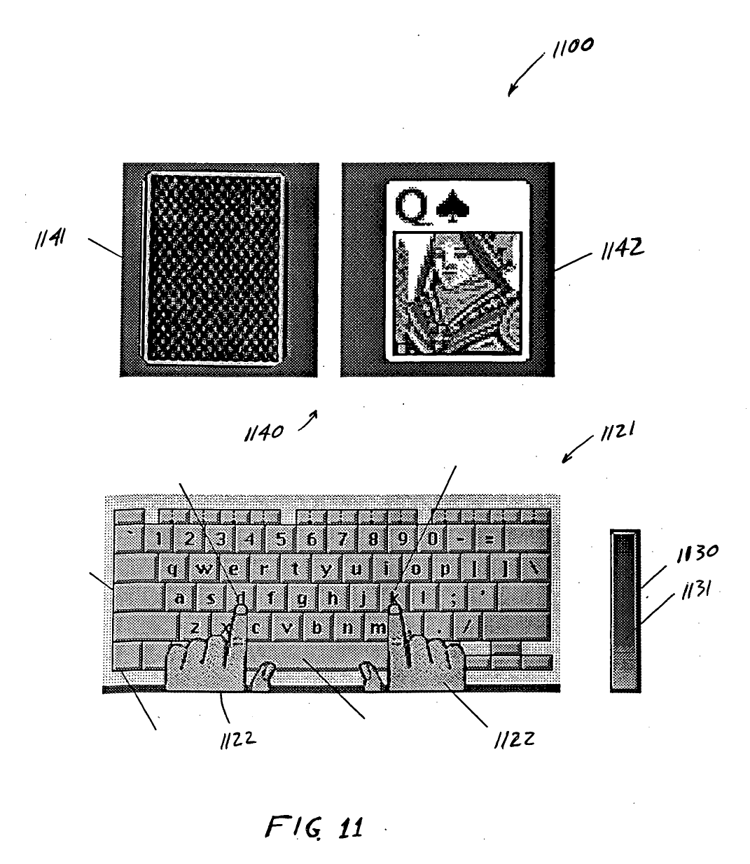

FIG. 11 is a simplified diagram illustrating a start configuration displayed by a system and method of testing cognitive function.

During the simulation phase (FIG. 5), a start configuration display 1100 may include a representation of a keyboard 1121 with overlying hands 1122. In accordance with some of test protocols, a response meter 1130 may provide feedback regarding speed of response during a portion of the test or simulation phase. The simulation phases for the exemplary tests include displaying cards 1140 in a particular manner and indicating how the test subject is expected to respond to the test trial event; in that regard, a relevant section of the display of the keyboard may be highlighted to indicate a proper response. Response meter 1130 may provide a further indication as to whether the user is responding sufficiently rapidly, and may generally be embodied in many forms known in the art such as a clock face, an hour glass, a dial or gauge, and the like.

In general, one or more decks of face-down cards 1141 may appear centrally on the display of the computer monitor. At some point in time (stimulus start, see, e.g. FIG. 7), one or more cards 1142 may turn face-up on top of or beside the deck. As described in detail above, each card may require a specific response or action on the part of the test subject, e.g. depressing one key of the keyboard. Depending upon the handedness of the test subject and other factors, for example, different keys may be designated as “true” or “false;” in some embodiments, keys may be selected to ensure that the dominant hand is used to answer the “true” condition. More complicated tests may be devised which require interaction with a number of keys (greater than two or three, for example) if appropriate for the test protocol and the cognitive ability being evaluated.

As noted above, representation of the keyboard 1121 may appear during the instruction phase; additionally, such a representation may also reappear after a run of consecutive incorrect responses. Additionally, visual feedback may vary depending, in part, upon whether a given response was correct or incorrect; for example, cards 1142 may be depicted as returning to, or being reinserted into, the deck 1141 in a different way for a correct response than for an incorrect response. In some testing embodiments, an incorrect response may also elicit a sound.

The following test protocols are provided by way of example only, and not by way of limitation. It will be appreciated by those of skill in the art that various modifications and alterations are within the scope and contemplation of the present disclosure.

1. Keyboard Key Test

Aim: To train the subject in response accuracy and speed using the keyboard.

The keyboard and response meter appear with keys used for response input outlined in red; various keys are easily used in combination. These keys may initially flash sequentially twice to attract attention to them, and then the hands 1122 appear and slide into the correct hand position. Specific keys then highlight in a random order. The subject is expected to press the highlighted key as quickly as possible. The keys may highlight every 1500-1700 ms. Graphics 1131 associated with response meter 1130 may rise at a steady rate to provide an indication that the user should respond. Response meter 1130 may stop responsive to user input; the test subject may inspect the color of the graphic 1131 as a measure of response speed. Other embodiments may use dials or other visual timing indicators. For an incorrect response (e.g. the wrong key is depressed or the timer expires), an error buzzer may sound. No cards appear in this test, and the keyboard representation remains throughout the subtest.

The test continues until each key has been depressed three times each, or at least once each and a total of nine keys have been correctly pressed, or a total test time elapses (60 seconds, for example). Anticipatory and post-stimulus feedback errors (key responses) are also recorded.

Trial settings:
Total required successes=9
Stimulus Start=1500 ms
Stimulus Stop time=0 ms
Feedback duration=200 ms
Post-ISI random range=0-200 ms
Minimum reaction time start=1600 ms
Maximum time for trial=5000 ms

2. Simple Reaction Test.

Aim: to test simple reaction time as a baseline for other cognitive reaction time tests.

Instruction Phase: A simulation first shows what the subject is expected to do. Initially, the start configuration depicts a keyboard (without hands) with the space bar key outlined in red, a central deck of cards face-down, and a response meter. At random intervals (between 1500-2500 ms, for example) a card appears face-up and the space bar key highlights (this may or may not be accompanied by an aural cue such as key-pressing sound or click). The subject may respond by pressing the correct key (i.e. the space bar in this example). Failure to respond, or depression of an incorrect key, results in a buzzer sound accompanied by an illustration of a yellow shadowed arrow; the arrow appears from the base, of the face-up card and extends to the space bar key. The arrow pointing to the space bar may serve as an indication that the space bar should be pressed as soon as the card turns face-up. This repeats for a total of three times before the instruction phase ends and the test phase begins. The subject may abort the simulated instruction by depressing the escape key, for example, or entering another cancel sequence.

Testing Phase: Testing may be in exactly the same format as the instruction phase, though the subject must respond and keyboard key highlighting is delayed. The appearance of one or two hands may indicate that the subject should prepare to start responding. In addition, the card deck, keyboard, and response meter may disappear briefly and redraw.

A single deck of face-down cards then appears centrally; this may or may not occur concomitantly with a shuffling sound. After a variable period (between 1500-2500 ms, for example), a randomly selected card appears face-up on top of the central deck. If the subject does not respond during a predetermined period of time, the space bar key highlights until a key is depressed. A reaction time is then recorded and visual feedback commences. Visual feedback for this test includes: the space bar key unhighlights; if the subject provided a proper response, the card moves to the right, turning over to face-down, and slides underneath the deck; or, if an incorrect key was depressed, the card moves to the left initially, and an error buzzer sounds. The test trials repeat with an ISI varying between 1500-2500 ms; initially, the same card is displayed for a number of test trials. If the trial takes longer than 5000 ms, then the error feedback occurs whether or not the subject responds.

The keyboard disappears after three consecutive correct trial responses, and will reappear after three consecutive incorrect responses. The test ends when twelve correct responses have been provided for this same card and a further three correct responses have been provided to subsequent randomly presented cards, or a total test time of sixty seconds elapses, whichever first occurs. Hence, after twelve correct responses, the cards displayed begin randomly to change, ensuring that the subject is aware of the importance solely of responding when a card turns face-up, i.e. the card value and suit are not relevant to the test.

This simple reaction time test may be repeated two other times throughout the entire test sequence or battery (for example, once after the combined monitoring task and once at the very end after the associate learning task) in order to determine whether the subject is fatiguing or concentrating more poorly as the test goes on.

Trial Settings:
Total required successes=12 (+3 extras)
Stimulus Start=1500 ms
Stimulus Stop time=0 ms
Feedback duration=200 ms
Post-ISI random range=0-1000 ms
Minimum reaction time start=1600 ms
Maximum time for trial=5000 ms

3. Choice Reaction Test

Aim: To assess a subject's efficiency in a simple choice reaction task, in this instance, choosing between red and black alternatives. Adding this simple choice component is expected to increase reaction time by approximately 50-150 ms.

Instruction Phase: A simulation first shows what the subject is expected to do. Initially, the start configuration depicts a keyboard (without hands) with the true and false keys outlined in red and a central deck of cards face-down; this appearance is very similar to the simple reaction time task just completed. At random intervals between 1500-2500 ms, a card appears face-up and the correct response key highlights accompanied by an additional key pressing sound or click. The subject may respond by pressing the correct key. Failure to respond, or depression of an incorrect key, results in a buzzer sound accompanied by an illustration of a yellow shadowed arrow; the arrow appears from the base of the face-up card and extends to the correct key, indicating which key should be depressed when a particular card appears face-up. The cards in the instruction phase are not proper cards, but contain red or black color filled rectangles. These are randomized in order of presentation during the simulation; the instruction phase continues until at least two cards of each color have been presented, and then the testing phase begins.

Testing Phase: The testing may be in exactly the same format as the instruction phase, using normal appearing playing cards, though the subject must respond and keyboard key highlighting is delayed. The appearance of the hands indicates that the subject should prepare to start responding. In addition, the card deck and keyboard disappear briefly and redraw.

A single deck of face-down cards then appears centrally; again, this may occur concomitantly with a shuffling sound. After a variable period between 1500-2500 ms, a randomly selected face-up card appears on top of the central deck. If the subject fails to respond within a predetermined time period, the correct true/false key highlights until a key is depressed. A reaction time is then recorded and visual feedback commences. Visual feedback for this test includes: the correct key unhighlights; if the subject depressed the correct key, the card moves to the right, turning over to face-down, and slides underneath the deck; or, if the subject depressed an incorrect key, the card moves to the left initially, and an error buzzer sounds. Test trials repeat, always showing a randomly selected card, with the ISI varying between 1500-2500 ms. If a trial takes longer than 5000 ms, then the error feedback occurs whether or not the subject responds.

The keyboard disappears after three consecutive correct trial responses, and will reappear after three consecutive incorrect responses. The test ends when the subject provides fourteen correct responses to either red or black cards, or after a total test time of sixty seconds has elapsed, whichever first occurs.

Trial Settings:
Total required successes=7 blacks+7 reds (or total of 14)
Stimulus Start=1500 ms
Stimulus Stop time=0 ms
Feedback duration=200 ms
Post-ISI random range=0-1000 ms
Minimum reaction time start=1600 ms
Maximum time for trial=5000 ms

4. Congruent Test

Aim: To assess a subject's efficiency in a more complex choice reaction task, in this instance, choosing between congruent card suit colors when confronted by two face-up cards placed vertically. Adding this more complex choice component is expected further to increase reaction time by approximately 50-150 ms over the choice reaction time. Recordation of data for tests of increasing complexity allows a regression line to be constructed showing increasing reaction time with increasing stimulus demands.

Instruction Phase: A simulation first shows what the subject is expected to do. Initially, the start configuration depicts a keyboard (without hands) with the true and false keys outlined in red and a central deck of cards face-down; the central deck then splits, sliding another deck of face-down cards adjacent the first. This appearance is similar to the choice reaction time task just completed, though the test layout differs by an extra deck of face-down cards.

At random intervals between 1500-2500 ms, two cards appear face-up, one on each respective deck, and the correct response key highlights. The subject may respond by pressing the correct key. Failure to respond, or depression of an incorrect key, results in a buzzer sound accompanied by an illustration of a yellow shadowed arrow; the arrow appears from the base of one of the face-up cards and extends to the correct key, indicating which key should be depressed when this particular combination of cards appears face-up. The cards in the simulation are not proper cards, but the same red or black color filled rectangle cards used in the choice reaction time task. The presentation of these is again randomized during the instruction phase (i.e. whether two congruent or different color cards). The instruction phase continues until at least two of each configuration have been presented, and then the testing phase begins.

Testing Phase: The test may be in exactly the same format using normal playing cards, though the subject must respond and arrows may not appear. The appearance of hands indicates that the subject should prepare to start responding. In addition, the card decks and keyboard disappear briefly and redraw. The dual decks of face-down cards appear again centrally concomitantly with a shuffling sound.

After a variable period of between 1500-2500 ms, randomly selected face-up cards appear, one on top of each respective deck, simultaneously. After a delay, the correct true/false key highlights until the subject provides a response. A reaction time is then recorded, and visual feedback commences. Visual feed back in this test includes: the correct key unhighlights; if the subject depressed the correct key, both cards move to the right, turning over to face-down, and sliding underneath their respective decks; or, if the subject depressed an incorrect key, the cards move to the left initially with an error buzzer sounding. Test trials repeat, always showing randomly selected cards, with the ISI varying between 1500-2500 ms. If a given trial takes longer than 5000 ms, then the error feedback occurs whether or not the subject has responded.

The keyboard disappears after three consecutive correct trials, and will reappear after three consecutive incorrect responses. The test ends when fourteen correct responses have been provided to either congruent or non-congruent card pairs, or a total test time of sixty seconds elapses, whichever first occurs.

Trial Settings:
Total required successes=7 congruent+7 non-congruent (or total of 14)
Stimulus Start=1500 ms
Stimulus Stop time=0 ms
Feedback duration=200 ms
Post-ISI random range=0-1000 ms
Minimum reaction time start=1600 ms
Maximum time for trial=5000 ms

5. Continuous Monitoring Test

Aim: This is the first of three linked tests. It measures vigilance and is a continuous performance task. The test trains subjects in an expectant monitoring task which is later combined with another choice decision task in order to test divided attention. A proper response comprises depressing the space bar when any card touches a white line. The white lines are horizontally placed equidistantly above and below the original face-down pack's location vertically.

Instruction Phase: A simulation first shows what the subject is expected to do. Initially, the start configuration depicts the keyboard (without hands) with the space bar outlined in red, five vertically centered face-up cards, and two horizontal lines. One horizontal line is disposed above the five cards, and one horizontal line is disposed below the five cards on the display. The cards move up and down, oscillating continuously in a seemingly random manner.

Individual cards may migrate progressively upward on the display at any point in time, hover in the same approximate location, or migrate progressively downward. It is not possible to predict reliably which way a particular card will move. All cards are constantly moving, and at some point during the simulation, one of the cards touches either the upper or the lower limiting line. The subject may respond by pressing the space key.

Failure to respond, or depression of an incorrect key, results in a buzzer sound. At this point in the instruction, the cards stop moving and a yellow arrow appears from the bottom of the card which is touching a line and extends to the space bar, which highlights simultaneously. After a brief delay, the instruction continues and the card which was touching the line becomes centered vertically and the space bar unhighlights. The instruction phase continues until at least one card has touched the upper line and at least one card (not necessarily the same card) has touched the lower line. The cards displayed during the instruction phase are proper cards.

Testing Phase: The test proper is exactly the same format, though the subject must respond and the keyboard key highlighting is delayed. The appearance of the hands indicates that the subject should prepare to start responding. In addition, the representations of the cards and keyboard disappear briefly and redraw.

At the beginning of a test trial, the five face-up cards appear again, centered vertically, concomitantly with a shuffling sound. The cards begin moving in the pseudo-random oscillations described above. After a variable period, one card will touch a line, representing the event which should elicit a response from the subject. If the subject does not provide a response after a predetermined duration, the space bar key highlights.

The card which has touched a line will travel away from the center of the display (but no further than a half a vertical card dimension beyond the upper or lower white line) such that it is no longer equivocal as to whether the line has been crossed. The card may continue migrating away from the center or may remain at the maximum allowed limit until the subject provides a response. A reaction time is then recorded, and visual feedback commences. Visual feedback provided in this test generally includes: the space bar key unhighlights; if the subject correctly depressed the space bar, the errant card returns to the center of the display; or, if the subject failed to provide a correct response, an error, buzzer sounds and the errant card does not change position. Additionally, if the space bar key is depressed when no card is touching or beyond a line, the error buzzer also sounds and an anticipatory error is recorded.

If the subject does not respond after a card has been beyond a line for a specified duration (for example, greater than or equal to two seconds), then the card jumps back a half card distance towards the center, and moves steadily outward again. This particular feedback strategy aims to attract attention to persistently missed cards.

In this test, the cards move incrementally, with each increment characterized by a minimum of six pixels, for example; variable additional steps of 0-6 pixels per movement increment may also be included. One “favored” card (randomized to a different card when this favored card reaches a line) has an additional gain factor (±4 pixels) added to its movement. A positive gain factor biases movement towards the lower line; conversely, if the gain factor is negative, the card may be biased toward the upper line. The keyboard disappears after three consecutive correct trials, and will reappear after three consecutive incorrect responses.

The test ends when the subject correctly responds to fourteen different line touching events (with respect to either upper or lower migrating cards) or when a total test time of sixty seconds has elapsed, whichever first occurs.

Trial Settings:
Total required successes=14
Stimulus Start=0 ms
Stimulus Stop time=0 ms
Feedback duration=0 ms
Post-ISI random range=0 ms
Minimum reaction time start=0 ms
Maximum time for trial=99999999 ms (about 27.8 hours)

6. One-Back Test

Aims: This is the second of the three tests designed to assess divided attention. This test provides a working memory task in which the subject must remember the prior card when responding; it is termed a “one-back” test because the subject is required to remember only one previous card, i.e. the last presented. This is also a training task for the next combined test.

Instruction Phase: A simulation first shows what the subject is expected to do. Initially, a start configuration illustrates a keyboard (without hands), with the true and false keys outlined in red, and a single central deck of cards face-down adjacent the keyboard. This appearance is similar to the choice reaction time task. At random intervals between 1500-2500 ms, a card appears face-up on the deck, and the correct response key highlights. The subject may respond by pressing the correct key.

Failure to respond, or depression of an incorrect key results in a buzzer sound accompanied by the appearance of a yellow shaded arrow; the arrow appears from the base of the face-up card and extends to the correct key, indicating which key is the proper response for the type of card displayed. The cards in the simulation are proper cards. The rule is based on whether the face-up card is the same, or has the same value, as the previous face-up card.

The instruction continues through all possible variations for consecutive cards. When the presently displayed face-up card is the same, or has the same value, for example, as the most recently displayed card, the true key is highlighted; conversely, when consecutive cards are different, the false key is highlighted. Several iterations through the foregoing instruction procedure should be sufficient for most subjects to work out the rules for responding, though this is not as easy as the prior tests. The instruction phase continues until at least one of each of the possible sequences has appeared, and then the testing phase begins.

Testing Phase: The testing phase may be in exactly the same format as the instruction phase, i.e. using normal playing cards; the subject must respond and keyboard key highlighting is delayed. The appearance of the hands indicates that the subject should prepare to start responding. In addition, the representation of the card deck and keyboard disappear briefly and redraw concomitantly with a shuffling sound.

After a variable period of between 1500-2500 ms, for example, a randomly selected face-up card appears. After a delay, the correct true or false key highlights if the subject does not provide a response by depressing a key. A reaction time is then recorded, and visual feedback commences. Visual feedback associated with this test may include the following: the correct key unhighlights; if the subject responded by depressing the correct key, the card moves to the right, turning over to face-down and sliding underneath the deck; or, if the subject depressed an incorrect key, an error buzzer may sound as the card moves to the left initially. Testing trials having the foregoing sequence repeat, always displaying randomly selected cards, with the variable ISI varying between 1500-2500 ms. If the trial lasts longer than a predetermined maximum trial duration, 5000 ms, for example, then the error feedback occurs whether or not the subject has provided a response.

The representation of the keyboard disappears after three consecutive correct trial event responses, and will reappear after three consecutive incorrect responses. The testing phase ends when the subject has correctly responded to fourteen different trial events, i.e. either sequential paired or non-paired cards, or when a total test time of ninety seconds has elapsed, whichever first occurs.

Trial Settings:
Total required successes=14
Stimulus Start=1500 ms
Stimulus Stop time=0 ms
Feedback duration=200 ms
Post-ISI random range=0-1000 ms
Minimum reaction time start=1600 ms
Maximum time for trial=5000 ms

7. Combined Monitoring/One Back test

Aims: This is the combination of tests five and six described above, and aims to assess divided attention. The test provides a difficult task in which errors or prolonged reaction times are likely to be common. The subject must perform the one-back task occurring in the center card, whilst observing a total of five cards as each jitters between two white horizontal lines.

Instruction Phase: There is no specific simulation or instruction component to this test, since the necessary instructions have already been provided in the previous two tests. In cases where the test is conducted or administered in isolation, however, it may be desirable to provide an instruction phase combining the rules presented above.

The One-Back task continues from the previous test; additionally, the start configuration includes horizontal lines, and further displays the jittering vertical movement of a single central card. After several correct responses are recorded, four other (peripheral) jittering cards appear on either side of the first as in the Monitoring task. These four peripheral cards do not change, nor are their denominations important in the test. The display does not include a representation of a keyboard for guidance. When the testing phase begins, the subject is expected to remember which keys must be used from the previous tests.

Testing Phase: The testing phase continues using the same format as the previous tests. After a variable period, one or more cards will touch a white line. The card touching the line will subsequently travel away from the center of the display (but no further than half a vertical card dimension beyond the line) so that it is no longer equivocal as to whether the line has been crossed. The card continues to migrate away from the center, or remains at the maximum allowed limit, until the subject responds by depressing the space bar.

A reaction time is then recorded as for the monitoring task, and visual feedback commences as set forth above. If the subject has correctly depressed the space bar key, the errant card or cards return to the center of the display. If an incorrect key is depressed (e.g. a key which is not relevant to the one-back task), an error buzzer sounds and the errant card does not change position. In addition, if the space bar key is depressed when no card is making contact with a line, the error buzzer will sound and an anticipatory response error is recorded. If the subject does not respond after a card has been beyond a line for a predetermined period (e.g. greater than or equal to two seconds), then the card touching the line jumps back a half card distance towards the center of the display and again begins to migrate outwards. This particular visual feedback strategy aims to attract attention to persistently missed cards.

As noted above, the cards may move incrementally; the movement of the cards in this test may be substantially similar to the movement described above with reference to the monitor test.

The one-back task portion of the testing phase executes simultaneously and independently, using normal appearing playing cards. After a variable period of between 1500-2500 ms, for example, the central face-down card turns face-up, revealing a randomly selected card; the display of the card remains until either the true or the false key is depressed. A reaction time is then recorded, and visual feedback commences substantially as described above with reference to the one-back test.

Test trials repeat, always displaying randomly selected cards, with the ISI varying between 1500-2500 ms, for example. If the card remains face-up for longer than a predetermined period of time (for example, 5000 ms), then the error feedback occurs whether or not the subject has responded.

A representation of the keyboard may appear after three consecutive incorrect responses. The testing phase ends when the subject has correctly responded to fourteen test trial events in both of the two tested tasks, or after a total test time of ninety seconds has elapsed, whichever first occurs.

Trial Settings:
Total required successes=14 one-back and 14 line-crossings
Stimulus Start=1500 ms
Stimulus Stop time=0 ms
Feedback duration=200 ms
Post-ISI random range=0-1000 ms
Minimum reaction time start=1600 ms
Maximum time for trial=5000 ms

8. Paired Card Matching Test (with Incidental Memory)

Aims: To assess speed and accuracy with respect to matching skills. Six pairs of different cards appear above dual decks of face-down cards; these six pairs comprise a legend. Cards appear face-up on these decks, and the subject must decide whether the face-up cards are part of the six pair legend or not. After the cards have been matched multiple times, incidental learning of these pairs is tested. No feedback occurs during this memory testing phase. It is expected that subjects with poor retentive memory abilities will do particularly poorly on the incidental memory component.

Instruction Phase: A simulation component first shows what the subject is expected to do. A start configuration initially displays a keyboard (without hands), with the true and false keys outlined in red, and a single central deck of cards face-down adjacent the keyboard. The deck splits in two and the second half slides adjacent the initial deck. Cards then flip and move upward on the display to form two rows of three card pairs centered horizontally above the face-down decks.

At random intervals between 1500-2500 ms, two cards appear face-up on the decks, and the correct response key highlights. The subject may respond by pressing the correct key. Failure to respond, or depression of an incorrect key results in a buzzer sound accompanied by the appearance of a yellow shadowed arrow; the arrow appears from the base of the face-up cards and extends to the correct key, indicating which key should be depressed responsive to the displayed combination of face-up cards. The cards in the simulation are proper cards.

The instruction test trials illustrate both true and false conditions. Hence, if a pair which is also represented in the six card legend appears, this is regarded as a true condition; conversely, a false condition occurs when a pair that is not represented in the six card legend appears. To facilitate learning of the pairs, no equivocal pairs (i.e. pairs having one of the two cards of the “true” legend pairs of cards) will ever appear during the instruction phase. Visual feedback differs for these conditions. For true pairs, the cards slide quickly to their matching cards. For false conditions, the cards turn face-over and slide underneath the decks. This feedback strategy should allow subjects to work out the rules for responding. Subjects are not shown an instructive simulation of the memory component. The instruction phase continues until at least two of each of the true and false conditions has appeared (facilitated if chance is taking too long), and then the testing phase begins.

Testing Phase: The test may be in the same format as the instruction phase, though the subject must respond, the keyboard key highlighting is delayed, and there are now six card pairs rather than three; accordingly, the legend in the testing phase comprises twelve cards arranged in six pairs. The appearance of the hands indicates that the subject should prepare to start responding. In addition, the representation of the card decks and the keyboard disappear briefly and redraw concomitantly with a shuffling sound.

Six card pairs are flipped over from the two face-down decks to indicate the set of cards which will be used as the legend for the testing phase. After a variable period of between 1500-2500 ms, for example, two randomly selected cards will be displayed face-up. After a specified delay, the correct true/false key highlights, and will remain highlighted until the subject provides a response by depressing a key. A reaction time is then recorded, and visual feedback commences as described above. If the response is incorrect, an error buzzer sounds.

The test subject may not be forewarned about the memory component of the test, though it is expected that after performing the test several times, the subject will be aware of the need to commit the legend's pairs to memory. Test trials repeat, with the ISI varying between 1500-2500 ms, until the each legend pair has been displayed twice, and non-legend pairs have been displayed at least six times. If a trial lasts longer than 5000 ms, then the error feedback occurs whether or not the subject has responded.

The representation of the keyboard disappears after three consecutive correct trials and may reappear after three consecutive incorrect responses. When the learning component has completed, the incidental memory component begins. The learning component may be timed, for example, such that the memory component is initiated if a specified time duration has elapsed (greater than or equal to eighty seconds, for instance).

During the memory component of the test, the legend disappears (or legend pairs turn face down) and card pairs continue to turn over. No error feedback is provided, and cards always flip over to the right and slide under their respective decks regardless of the key depressed. No error buzzer sounds. If a particular test trial takes longer than 5000 ms, then the error feedback occurs. About thirty successful responses are required to complete this test. Card pairs flip over to face-up, at intervals of approximately 1500-2500 ms, until either: all legend card pairs have been shown at least once, and a similar number of non-legend card pairs has been displayed; or a total of 150 seconds for both test components has elapsed.

Trial Settings:
Total required successes=12 legend pairs, 6 foils, and then 6 of each in memory component
Stimulus Start=1500 ms
Stimulus Stop time=0 ms
Feedback duration=200 ms
Post-ISI random range=0-1000 ms
Minimum reaction time start=1600 ms
Maximum time for trial=5000 ms

9. Associate Learning Test

Aims: This final test allows assessment of both learning and retentive memory, with a matching ability control test included. The test resembles the paired-card matching test in layout, except that all but one pair in the legend is face-down. The subject must remember the cards in the hidden, or face-down, pairs. The face-up pair can be matched directly by comparison without the need to remember it. This face-up pair is the control pair, since subjects with primary memory disorders should be able to match the control pair even though they cannot recall the hidden cards. Some subjects with feigned memory loss might be expected to have trouble with both hidden and displayed pair matching (beyond a chance level). This is a hard test which should be a good discriminator of memory and concentration ability; additionally, it is presented as the last test to maximize the detrimental effects of fatigue or poor concentration. It is also expected that subjects will not recall all four pairs correctly on the first presentation, but that a learning curve will occur such that errors are corrected with subsequent feedback.

Instruction Phase: A simulation first shows what the subject is expected to do. It is very similar to the paired-cards matching test. Initially, a start configuration displays a keyboard (without hands) with the true and false keys outlined in red, and a single central deck of cards face-down. The deck splits in two and the second half slides adjacent the initial deck. Cards then flip to face-up and move upward on the display to form two rows of three cards centered horizontally above the face-down decks.

At random intervals between 1500-2500 ms, two cards appear face-up on the decks and the correct response key highlights, substantially as described above. The instruction phase displays each of the three pairs to be remembered twice in succession, with foil presentations randomly interspersed. If the pair displayed face-up exists in the legend, then the true key should be selected. When the subject responds in the initial instruction phase, the pair of cards slides upward in the display to join the legend above the representation of the dual decks of cards. After first presentation of a legend's pair of cards during the instruction, the presented legend pair's cards will turn to face-down such that when all legend card pairs have been shown, the two outer pairs will be mainly face-down, whilst the central pair is face-up throughout.

Once a pair of cards has turned over after the subject has entered a response, the correct key unhighlights and visual feedback occurs. If the displayed card pair is part of the set to be remembered, and that legend card pair is already face-down, the matching card pair in the legend flip to face-up, and the stimulus cards slide to their matching grid position (from left to right) so the subject can see that the cards are the same as the new pair; after a brief delay of about 0.5 seconds, for example, the cards flip over in-situ so they are face-down. The central pair in the legend, however, never turns face-down, though the other aspects of the visual feedback provide sufficient instruction.

Once all the simulation card pairs (six cards) have appeared twice, the instruction phase presents random pairs of cards such that either a pair matching the legend or a pair not matching the legend appears. If a displayed pair matches a pair in the legend, the true key highlights. The subject may respond by depressing the correct key. Failure to respond, or depression of an incorrect key results in a buzzer sound, and the correct key is highlighted, for example, by an arrow. The arrow may then be removed, the legend cards flip to face-up, and the cards of the displayed pair slide to their correct positions. If a displayed pair does not exactly match any of the legend's pairs, then the false key is highlighted and the cards of the pair flip over to and then slide underneath their respective decks.

Both unequivocal and equivocal foils (i.e. with neither or one only of the cards of a true legend pair, respectively) in any order can occur so that the subject must truly recall both cards in each legend pair to be completely accurate. The instruction and simulation strategy should allow subjects to work out the rules for responding. The instruction phase continues until at least two of each of the true and false conditions has appeared after all legend cards have been laid out, and then the testing phase begins.

Testing Phase: The test may be in substantially the same format, though the subject must respond, the key highlighting is delayed, and there will now be five card pairs (i.e. four face-down card pairs with a centrally placed face-up pair). The appearance of the hands indicates that the subject should prepare to start responding. In addition, the representation of the card decks and the keyboard disappear briefly and redraw concomitantly with a shuffling sound; the legend disappears completely.

As in the instruction phase, the legend is built by flipping all card pairs in the legend to their grid positions and then displaying a new pair of cards. A card pair may then be displayed after a variable period of between 1500-2500 ms. Card pairs are selected randomly so that no cards are repeated and no pair is the same from trial to trial. After a delay following display of the card pair, the correct true/false key highlights and remains so until the subject enters a response.

A reaction time is then recorded, and visual feedback commences as discussed above with reference to the instruction phase; during the testing phase, however, once turned face-down, the corresponding face-down pairs in the legend are not flipped over during the feedback. The displayed card pair moves to the appropriate pile, and then flips to face-down. If the displayed pair is not part of the legend, the cards flip over and slide beneath their respective decks. If the response input was incorrect, an error buzzer sounds.

Test trials repeat, always showing randomly selected card pairs (either matching a pair of cards in the legend or not), with the ISI varying between 1500-2500 ms, until each of the legend pairs have been displayed five times and non-legend pairs an equal number of times. If an individual trial lasts longer than a specified period (5000 ms, for example), then the error feedback occurs whether or not the subject provides a response. The representation of the keyboard disappears after three consecutive correct responses and may reappear after three consecutive incorrect responses. The test ends if more than four minutes elapses.

Trial Settings:
Total required successes=20 legend pairs, 20 non-legend pairs
Stimulus Start=1500 ms
Stimulus Stop time=0 ms
Feedback duration=200 ms
Post-ISI random range=0-1000 ms
Minimum reaction time start=1600 ms
Maximum time for trial=5000 ms

Individual tests, or an entire test battery or sequence, may be cancelled at any time using predetermined commands. The subject may be warned that data will be lost if the test or tests are canceled. Upon completion of a test sequence, as noted above, the subject may be prompted to transmit test data and results to a central server for analysis.

Normative data may be collected. Simple descriptive statistics may compute mean responses or scores, as well as variability measures regarding the mean, for all tests administered; accordingly, an indication or measure of psychomotor speed and consistency may be computed. Additionally, some test data may be grouped to enable across test comparisons.

The present invention has been illustrated and described in detail with reference to particular embodiments by way of example only, and not by way of limitation. Those of skill in the art will appreciate that various modifications to the disclosed embodiments are within the scope and contemplation of the invention as set forth herein. Therefore, it is intended that the invention be considered as limited only by the scope of the appended claims.

1. A method of evaluating cognitive function; said method comprising:
administering a test operative to diagnose cognitive impairment; and
instructing a subject regarding rules for said test without providing cultural cues.

2. The method of claim 1 wherein said instructing comprises minimizing language-based cues.

3. The method of claim 1 wherein said instructing comprises simulating a test trial event.

4. The method of claim 3 wherein said instructing further comprises indicating a proper response to said test trial event.

5. The method of claim 1 wherein said administering and said instructing comprise utilizing a computerized system.

6. The method of claim 1 wherein said administering comprises recording responses to test trial events.

7. The method of claim 6 wherein said administering further comprises recording data related to said responses.

8. The method of claim 7 further comprising analyzing said responses and said data relative to previously recorded data records.

9. The method of claim 8 wherein said previously recorded data records are obtained during a previous administration of a test.

10. The method of claim 8 wherein said previously recorded data records are normative data for a population.

11. The method of claim 7 further comprising transmitting said responses and said data to a remote device.

12. The method of claim 1 wherein said administering comprises providing a plurality of tests administered in sequence.

13. The method of claim 12 wherein said instructing comprises simulating a test trial event for each of said plurality of tests.

14. The method of claim 8 wherein said analyzing comprises identifying pre-symptomatic cognitive impairment.

15. The method of claim 1 further comprising selectively repeating said administering and said instructing for a plurality of discrete tests.

16. A method of administering a sequence of tests; said method comprising:
selecting a test; said test comprising a plurality of test trials and operative to diagnose a condition of cognitive impairment;
instructing a subject regarding rules for responding to said plurality of test trials without providing cultural cues;
administering said test;
recording responses to a plurality of test trials displayed during said administering; and
selectively repeating said identifying, said instructing, said administering, and said recording for an additional test.

17. The method of claim 16 wherein said instructing comprises minimizing language-based cues.

18. The method of claim 16 wherein said instructing comprises simulating at least one of said plurality of test trials.

19. The method of claim 18 wherein said instructing further comprises indicating a proper response to said one of said plurality of test trials.

20. The method of claim 16 wherein said selecting, said instructing, said administering, and said recording comprise utilizing a computerized system.

21. The method of claim 16 wherein said recording further comprises recording data related to said responses.

22. The method of claim 21 further comprising analyzing said responses and said data relative to previously recorded data records.

23. The method of claim 22 wherein said previously recorded data records are normative data for a population.

24. The method of claim 21 further comprising transmitting said responses and said data to a remote device.

25. The method of claim 22 wherein said analyzing comprises identifying pre-symptomatic cognitive impairment.

26. An apparatus comprising:
a testing module operative to administer a test; and
an instruction module operative to instruct a subject regarding rules for said test without providing cultural cues.

27. The apparatus of claim 26 wherein said instruction module instructs said subject without providing language-based cues.

28. The apparatus of claim 26 wherein said instruction module comprises a test simulator operative to provide a simulation of a test trial event and to provide an indication of a proper response to said test trial event.

29. The apparatus of claim 26 wherein said testing module and said instruction module are implemented in computer software.

30. The apparatus of claim 26 further comprising a data structure operative to record responses to test trial events.

31. The apparatus of claim 30 wherein said data structure is further operative to record data related to said responses.

32. The apparatus of claim 31 further comprising an analytic module operative to analyze said responses and said data relative to previously recorded data records.

33. The apparatus of claim 31 further comprising a network interface allowing transmission of said responses and said data to a remote device.

34. The apparatus of claim 26 wherein said testing module is operative to administer a plurality of tests in sequence.

35. The apparatus of claim 34 wherein said instruction module is operative to simulate a test trial event for each of said plurality of tests.

36. The apparatus of claim 32 wherein said analytic module comprises a performance evaluator operative to identify test trial responses and data indicative of pre-symptomatic cognitive impairment.

37. A computer readable medium encoded with data and computer executable instructions; the data and instructions causing an apparatus executing the instructions to:
identify a test operative to diagnose a condition of cognitive impairment;
instruct a subject regarding rules for said test without providing cultural cues; and
administer said test to said subject.

38. The medium of claim 37 further encoded with data and instructions and further causing an apparatus to identify and to administer a plurality of discrete tests in sequence; and wherein the apparatus is further caused to instruct a subject regarding the rules for each of said plurality of discrete tests.

39. The medium of claim 37 further encoded with data and instructions and further causing an apparatus to instruct a subject without providing language-based cues.

40. The medium of claim 37 further encoded with data and instructions and further causing an apparatus to:
simulate a test trial event; and
indicate a proper response to said test trial event.

41. The medium of claim 37 further encoded with data and instructions and further causing an apparatus to record responses to test trial events.

42. The medium of claim 41 further encoded with data and instructions and further causing an apparatus to record data related to said responses.

43. The medium of claim 42 further encoded with data and instructions and further causing an apparatus to analyze said responses and said data relative to previously recorded data records.

44. The medium of claim 43 further encoded with data and instructions and further causing an apparatus to transmit said responses, said data, and analytic results based thereupon to a remote device.

45. The medium of claim 43 further encoded with data and instructions and further causing an apparatus to identify pre-symptomatic cognitive impairment.

46. A method of evaluating the efficacy of a treatment regimen for treating cognitive impairment; said method comprising:
selecting a test operative to evaluate cognitive function;
instructing a subject regarding rules for said test without providing cultural cues;
administering said test;
recording responses to a plurality of test trials displayed during said administering;
responsive to said recording, measuring a condition of cognitive impairment;
treating said subject in accordance with a treatment regimen;
selectively repeating said selecting, said instructing, said administering, said recording, and said measuring; and
responsive to said selectively repeating, evaluating said treatment regimen using a comparison of results obtained during said measuring.

47. The method of claim 46 wherein said instructing comprises minimizing language-based cues.

48. The method of claim 46 wherein said instructing comprises:
simulating a plurality of test trials; and
indicating a proper response to each of said plurality of test trials.

49. The method of claim 46 wherein said selecting, said instructing, said administering, said recording, and said measuring comprise utilizing a computerized system.

50. The method of claim 46 wherein said recording further comprises recording data related to said responses.

51. The method of claim 50 wherein said measuring comprises analyzing said responses and said data relative to previously recorded data records.

52. The method of claim 46 wherein said treating comprising administering a cognition enhancing drug.

53. A system of evaluating cognitive function; said system comprising:
a testing module operative to administer a test;
an instruction module operative to instruct a subject regarding rules for said test without providing cultural cues; and
a test coordinator operative to control operation of said testing module and said instruction module in accordance with a test protocol.

54. The system of claim 53 wherein said instruction module instructs said subject without providing language-based cues.

55. The system of claim 53 wherein said instruction module comprises a test simulator operative to provide a simulation of a test trial event and to provide an indication of a proper response to said test trial event in accordance with instructions from said test coordinator.

56. The system of claim 53 wherein said testing module, said instruction module, and said test coordinator are implemented in computer software.

57. The system of claim 56 wherein said test coordinator is implemented at a first device and said testing module and said instruction module are implemented at a second device coupled to said first device by a network connection.

58. The system of claim 53 further comprising a data structure operative to record responses to a plurality of test trial events and data related to said responses.

59. The system of claim 58 further comprising an analytic module operative to analyze said responses and said data relative to previously recorded data records.

60. The system of claim 53 wherein said test coordinator is operative:
to instruct said testing module to administer a plurality of tests in sequence; and
to instruct said instruction module to simulate a test trial event for each of said plurality of tests.

61. The system of claim 59 wherein said analytic module comprises a performance evaluator operative to identify test trial responses and data indicative of pre-symptomatic cognitive impairment.
